(19) United States
(12) Patent Application Publication
(10) Pub. No.: US 2023/0172425 A1
(43) Pub. Date: Jun. 8, 2023

(54) INFORMATION PROCESSING METHOD, ELECTRONIC DEVICE, AND COMPUTER STORAGE MEDIUM

(71) Applicants: Li Yan, Beijing (CN); NEC CORPORATION, Minato-ku (JP)

(72) Inventors: Yan LI, Beijing (CN); Li QUAN, Beijing (CN); Ni ZHANG, Beijing (CN)

(73) Assignee: NEC CORPORATION, Tokyo (JP)

(21) Appl. No.: 17/926,368

(22) PCT Filed: Apr. 26, 2021
(86) PCT No.: PCT/CN2021/090029

§ 371 Date: Nov. 18, 2022

(30) Foreign Application Priority Data
May 18, 2020 (CN) 202010421197.1

(51) Int. Cl. A61B 1/00

(52) U.S. Cl. CPC A61B 1/00009; A61B 1/00045; A61B 1/00059

(57) ABSTRACT

Provided are an information processing method, an electronic device, and a computer storage medium. The method comprises: receiving first information which is associated with an operation behavior of a medical testing device, wherein the first information is associated with data collection performed by the medical testing device during operation; and outputting at least part of the first information. By using the method, quality control can be performed on operation behaviors associated with examination, and the quality of a result obtained by an operation behavior, deviation from a recommended operation, a suggested modification direction and any possible statistical information can be presented, thereby a doctor can be helped in improving operations performed on a medical testing device.

### FIELD

Exemplary implementations of the present disclosure generally relate to the technical field of medical quality control, and more particularly, to an information processing method, an electronic device and a computer storage medium.

### BACKGROUND

Medical examination procedures performed on patients often involve complex manual operations. At present, the development of computer technology has provided more and more support for medical assistant operation. For example, for endoscopy, a doctor need to move the endoscope within the patient's body in order to acquire image data at multiple locations within the patient's body. There may be differences in operation of different doctors, for example, experienced doctors can complete a full endoscopy procedure independently, while inexperienced doctors may miss certain predetermined key site locations and/or cause discomfort to the patient due to inappropriate motion of the endoscope. Therefore, an effective technical solution is expected to provide medical assistance to guide operation of endoscopy.

In addition, with the further development of medicine, after doctors perform various medical tests such as endoscopy, it is expected to perform quality control on operation behaviors associated with the tests.

### SUMMARY

Exemplary Implementations of the present disclosure provide a technical solution of medical quality control.

In a first aspect of the present disclosure, there is provided an information processing method. The method comprises: receiving first information associated with an operation behavior of a medical testing device, the first information being associated with data collection performed by the medical testing device during operation; and outputting at least part of the first information.

In a second aspect of the present disclosure, there is provided an information processing method. The method comprises: receiving, at the terminal device, first identification information associated with a medical device; obtaining second identification information of an operator associated with the terminal device; and associating the medical device with the operator based on the first identification information and the second identification information.

In a third aspect of the present disclosure, there is provided an information processing method. The method comprises: receiving, at the terminal device, first indication information from a user, the first indication information indicating at least one operation performed by a medical device; receiving second indication information from the user, the second indication information indicating an operator of the at least one operation; and associating the indicated at least one operation with the indicated operator.

In a fourth aspect of the present disclosure, there is provided an electronic device. The electronic device comprises: at least one processing unit; at least one memory coupled to the at least one processing unit and storing instructions for execution by the at least one processing unit that, when executed by the at least one processing unit, cause the device to perform a method of any of the first, the second, and the third aspect.

In a fifth aspect of the present disclosure, there is provided a computer readable storage medium having computer readable program instructions stored thereon for performing a method according to any one of the first, the second, and the third aspect.

This Summary is provided to introduce a selection of concepts in a simplified form that are further described below in the BRIEF Description. This Summary is not intended to identify key features or essential features of the present disclosure, nor is it intended to be used to limit the scope of the present disclosure.

### BRIEF DESCRIPTION OF THE DRAWINGS

Through the following detailed description with reference to the accompanying drawings, the above and other objectives, features, and advantages of example embodiments of the present disclosure will become more apparent. In the exemplary implementations of the present disclosure, the same reference numerals usually refer to the same components.

FIG. 1 schematically shows a block diagram of a human environment in which an endoscopy may be performed according to an exemplary implementation of the present disclosure;

FIG. 2 schematically shows a block diagram of medical assistance operation according to an exemplary implementation of the present disclosure;

FIG. 3 schematically shows a flowchart of a medical assistance operation method according to an exemplary implementation of the present disclosure;

FIG. 4A schematically shows a block diagram of a motion model according to an exemplary implementation of the present disclosure;

FIG. 4B schematically shows a block diagram of a process of acquiring a motion model according to an exemplary implementation of the present disclosure;

FIG. 5 schematically shows a block diagram of a process of mapping a set of image sequences in an image sequence to a set of key site locations according to an exemplary implementation of the present disclosure;

FIG. 6 schematically shows a block diagram of a process for selecting an image associated with key site locations for storage according to an exemplary implementation of the present disclosure;

FIG. 7A schematically shows a block diagram of a data structure of a motion track according to an exemplary implementation of the present disclosure;

FIG. 7B schematically shows a block diagram of a process of providing a next destination location according to an exemplary implementation of the present disclosure;

FIG. 8 schematically shows a block diagram of a user interface providing medical assistance operation according to an exemplary implementation of the present disclosure;

FIG. 9 schematically shows a block diagram of another user interface providing medical assistance operations according to an exemplary implementation of the present disclosure;

FIG. 10 schematically shows a block diagram of medical assistance operation apparatus according to an exemplary implementation of the present disclosure;

FIG. 11 schematically shows a schematic diagram of a quality control environment that may be used to implement exemplary implementations of the present disclosure;

FIG. 12 shows a schematic diagram 1200 when a background management system is running;

FIG. 13 shows a schematic diagram 1300 when a client system is running;

FIG. 14 shows a schematic diagram 1400 when a client system is running;

FIG. 15 shows a schematic diagram 1500 when a client system is running;

FIG. 16 schematically shows a flowchart of an information processing method 1600 according to an exemplary implementation of the present disclosure;

FIG. 17 shows a schematic diagram 1700 when the client system is running;

FIG. 18 schematically shows a flowchart of an information processing method 1800 according to an exemplary implementation of the present disclosure;

FIG. 19 shows a schematic diagram 1900 when a client system is running;

FIG. 20 shows a schematic diagram 2000 when a client system is running;

FIGS. 21A-21B show schematic diagrams 2100-1-2100-2 when a client system is running;

FIGS. 22A-22C show schematic diagrams 2200-1-2200-3 when a client system is running;

FIG. 23 shows a schematic diagram 2300 when a client system is running;

FIGS. 24A-24B show schematic diagrams 2400-1-2400-2 when a client system is running;

FIGS. 25A-25E show schematic diagrams 2500-1-2400-5 when a client system is running;

FIG. 26 shows a schematic diagram 2600 when a client system is running;

FIG. 27 shows a schematic diagram 2700 when a client system is running;

FIGS. 28A-28E show schematic diagrams 2800-1-2800-5 when a client system is running;

FIGS. 29A-29E show schematic diagrams 2900-1-2900-5 when a client system is running;

FIGS. 30A-30E show schematic diagrams 3000-1-3000-5 when a client system is running;

FIGS. 31A-31B show schematic diagrams 3100-1-3100-2 when a client system is running;

FIG. 32 shows a schematic diagram 3200 when a client system is running;

FIG. 33 schematically shows a flowchart of an information processing method 3300 according to an exemplary implementation of the present disclosure;

FIG. 34 schematically shows a block diagram 3400 of an information processing apparatus 3410 according to an exemplary implementation of the present disclosure;

FIG. 35 schematically shows a block diagram of medical assistance operation apparatus according to an exemplary implementation of the present disclosure;

### DETAILED DESCRIPTION OF EMBODIMENTS

Preferred embodiments of the present disclosure will be described in more details below with reference to the drawings. Although the drawings illustrate preferred embodiments of the present disclosure, it should be understood, however, that the present disclosure may be embodied in various forms and should not be limited by the exemplary implementations set forth herein. On the contrary, the embodiments are provided to make the present disclosure more thorough and complete and to fully convey the scope of the present disclosure to those skilled in the art.

As used herein, the term “comprises” and its variants are to be read as open-ended terms that mean “comprises, but is not limited to.” The term “or” is to be read as “and/or” unless specifically stated otherwise. The term “based on” is to be read as “based at least in part on.” The terms “one example embodiment” and “one embodiment” are to be read as “at least one example embodiment.” The term “another example implementation” is to be read as “at least one additional example implementation”. The terms “first”, “second” and so on can refer to same or different objects. The following text also can comprise other explicit and implicit definitions.

Machine learning techniques have been applied to a variety of applications comprising medicine. Medical testing device often involves complex procedures, especially for endoscopy, where an endoscope needs to be inserted into a patient in order to capture images of various body locations. The inspection process needs to ensure that an image is acquired at a set of key site locations. The endoscope can move along different motion tracks according to the doctor's operation. Improper operation may lead to missing some key sites that should be tested. Therefore, how to provide medical assistance operations in a more effective way has become a research hotspot.

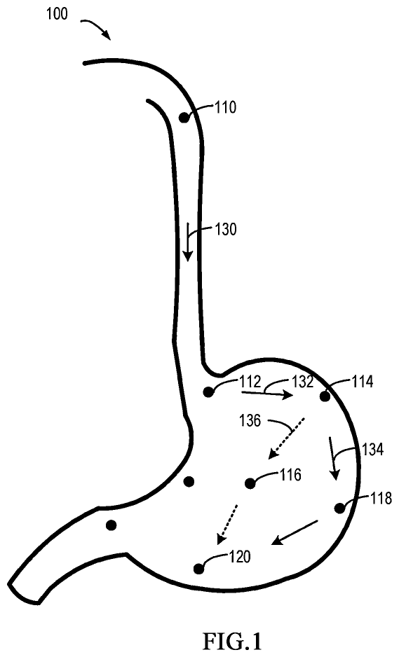

Endoscopy can be applied to the inspection of multiple human body parts. For example, it can be divided into esophagoscope, gastroscope, duodenum, colonoscopy and other types according to the body part. In the following, details of an exemplary implementation of the present disclosure are described by taking a gastroscope as an example. The application environment of an exemplary implementation of the present disclosure will be described first with reference to FIG. 1. FIG. 1 schematically shows a block diagram 100 of a human environment in which an endoscopy may be performed according to an exemplary implementation of the present disclosure. According to the endoscopic practice, the endoscope should reach a set of predetermined key site locations during the examination, and collect images at these key site locations to determine whether there is an abnormality at that locations. As shown in FIG. 1, during the process of inserting the endoscope into the human stomach, a plurality of key site locations 110, 112, 114, 116, 118, and 120 may be passed through.

The endoscope can first pass through the pharynx and reach a key site location 110, as indicated by an arrow 130, the endoscope can travel down the esophagus into the stomach and can reach a key site location 112. Further, as indicated by an arrow 132, the endoscope can reach a key site location 114. It will be appreciated that the endoscope can move in different directions within the stomach due to the large space inside the human body and due to the differences in how the doctor operates. For example, when the endoscope reaches the key site location 114, it may reach a key site location 118 in the direction indicated by an arrow 134, or it may also reach a key site location 116 in the direction indicated by an arrow 136. Although a set of key site locations has been defined in the operational specification, doctors can only adjust the motion track of the endoscope based on their own experience, and the motion track may only cover part of key site locations.

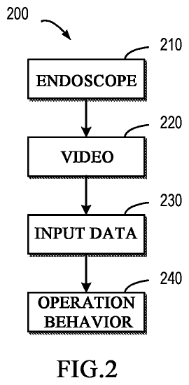

In order to at least partially solve the above-mentioned deficiencies in the endoscopy, according to an exemplary implementation of the present disclosure, a technical solution for a medical assistance operation apparatus is provided. The outline of the technical solution is first described with reference to FIG. 2. FIG. 2 schematically shows a block diagram 200 of medical assistance operation according to an exemplary implementation of the present disclosure. As shown in FIG. 2, as an endoscope 210 is inserted into a human body and moves therein, the endoscope 210 can capture a video 220 which can be viewed by the doctor in real time.

It will be appreciated that input data 230 (e.g., comprising a sequence of image data) may be obtained based on the video 220. For example, input data 230 may comprise one or more video clips, one video clip may comprise images of the endoscope 210 passing near a person's pharynx, and another video clip may comprise images of endoscope 210 passing near a person's esophagus. It will be understood that the format of the input data 230 is not limited in the context of this disclosure. For example, the input data 230 may be video data, a set of image sequences in a video in time sequence, or a plurality of image data with time information. According to an exemplary implementation of the present disclosure, the input data may be saved in the original video format, or may also be saved in a customized intermediate format.

It will be appreciated that the input data can be identified with a unique identifier. For example, a doctor ID and the time when the examination was performed may be used as an identifier, endoscope apparatus ID and the time when the examination was performed may be used as an identifier, a patient ID and the time when the examination was performed may be used as an identifier, or the above combination as a unique identifier. In turn, information related to an operation behavior 240 of the endoscope 210 may be determined based on the input data 230.

In this way, the doctor can be provided with effective medical assistance and instructed (especially inexperienced doctors) in their operations in order to avoid missing a certain/some key site location. Further, with the exemplary implementation of the present disclosure, the doctor can be guided to traverse all the key site locations as soon as possible, which can improve the efficiency of endoscopy, shorten the time the endoscope 210 is in the patient's body so as to reduce bad experience of a patient.

Specifically, the medical assistance operation can be provided in real time during the endoscopy performed by the doctor. Information related to operation behavior of the endoscope may be provided in real time based on a current location of the endoscope. For example, at least any one of the following may be provided in real time: a location of the key site where the endoscope is currently located, an image about the location of the key site, motion track the endoscope has passed, a next destination location of the endoscope, as well as statistics on endoscopy operation, and more. For example, the above-mentioned information can be displayed on a dedicated display device, alternatively and/or additionally, the above-mentioned information can also be displayed on a display device of the endoscopic apparatus.

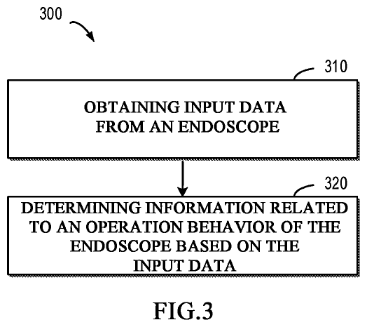

Hereinafter, more details of the medical assistance operation will be described with reference to FIG. 3. FIG. 3 schematically shows a flowchart of a medical assistance operation method 300 according to an exemplary implementation of the present disclosure. At block 310, input data 230 may be obtained from the endoscope 210. It will be appreciated that as the endoscope 210 moves within the body, input data may be acquired at different locations.

The input data 230 may be used to determine information or data required for endoscopic detection, and further, the input data 230 may also be used to determine information or data related to the operation behavior of the endoscope. Illustratively, the input data 230 may comprise image data collected at multiple locations during operation of the endoscope 210. It will be appreciated that the image data herein may be the original collected data, or may be processed (e.g., noise reduction processing, brightness processing, etc.) data. Based on acquisition frequency of the image acquisition device of the endoscope 210, the image data may comprise, for example, images at 30 frames per second (or other frame rate). It will be appreciated that in the context of the present disclosure, the format of the input data 230 is not limited.

The input data 230 herein may comprise at least any one of the following: video data, a set of image sequences arranged in time sequence, and a plurality of image data with time information. For example, the video data may comprise video stream formats and may support standards for multiple video formats. As another example, the image sequences may also comprise a series of individual images. At this time, the amount of the obtained input data 230 may gradually increase as the endoscopy is performed. For example, when the endoscope reaches the pharynx, an image sequence of the pharynx can be acquired; when the endoscope reaches the esophagus, a further image sequence of the esophagus can be acquired.

In addition, an identifier corresponding to the input data 230 may be further acquired or determined to identify the input data 230. Different identifications may distinguish one or a combination of one or more of the following: different patients, different examination times, different testing sites, and different testing operators.

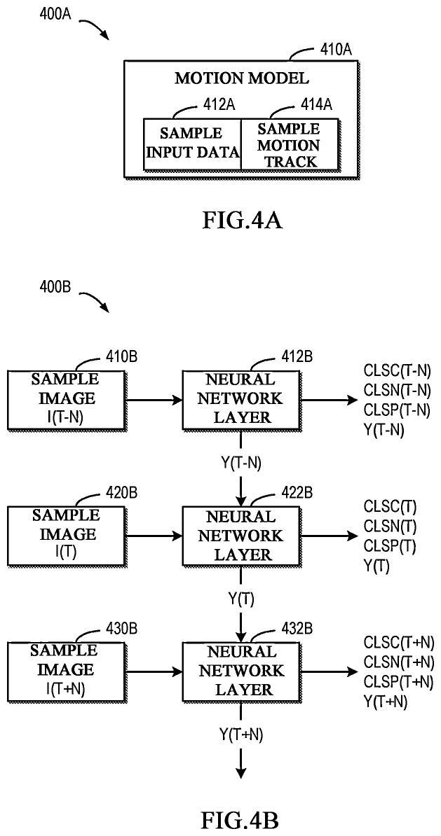

According to an exemplary implementation of the present disclosure, at block 320, information related to the operation behavior of the endoscope is determined based on the input data. The information can comprise various contents, for example, a current location of the endoscope, image data collected at the current location, motion track of the endoscope, a next destination location of the endoscope, and the statistical information of the input data, as well as statistics on the operation behavior, and more. In the following, more relevant details will be described with reference to FIGS. 4A and 4B.

By way of example, information related to the operation behavior of the endoscope may be determined according to the time series relationship of the input data 230. Furthermore, according to exemplary implementations of the present disclosure, various aspects of information related to the operation behavior 240 may be determined based on machine learning techniques and utilizing the input data 230. For example, it can be determined whether the current location, the motion track of the endoscope 210, and whether the motion track has reached a location of a key site desired to be inspected, and the like. Further, a destination location that should be reached in the next step can be determined. Specifically, a motion model 410A may be obtained based on machine learning technique using sample data collected during historical operation. FIG. 4A schematically shows a block diagram of the motion model 410A according to an exemplary implementation of the present disclosure. The motion model 410A may comprise an association between a sample input data 412A and a sample motion track 414A. Here, the sample input data 412A may be collected at a plurality of sample locations during the endoscopy examination, and a sample motion track 414A may comprise the motion track of the endoscope used to acquire the sample input data 412A.

It will be appreciated that the sample input data 412A and the sample motion track 414A herein may be sample training data used to train a motion model 410. According to an exemplary implementation of the present disclosure, a training session may be performed using the sample input data 412A and the corresponding sample motion track 414A. In the context of the present disclosure, one or more training sessions may be performed using sample training data from one or more endoscope examinations, respectively.

It will be appreciated that the above only schematically shows an example of the motion model 410A, and that other models may also be provided in accordance with exemplary implementations of the present disclosure. For example, another model may comprise associations between sample input data collected at multiple sample locations during endoscopy examination and corresponding key site locations for the multiple locations where the sample input data was acquired. With such a model, each image data in the input data 230 can be mapped to corresponding key site locations, respectively. Thus, based on the model and the input data, the location of key sites that the endoscope passes through can be determined. Further, based on the acquisition time of the image data and the locations of the above-mentioned key sites, the motion track of the endoscope can be determined.

Illustratively, training may be performed based on techniques such as Recurrent Neural Network (RNN), Long Short Term Memory (LSTM), etc. to obtain the motion model 410A. According to an exemplary implementation of the present disclosure, the motion model 410A may be obtained based on sample input data and corresponding sample motion tracks collected during historical examination using the training method described above. According to an exemplary implementation of the present disclosure, endoscopy operation can be performed by a doctor, and the above-described model can be trained using the collected data as a sample.

For example, motion of an endoscope can be manipulated by an experienced doctor according to an endoscopic procedure. At this time, sample motion track of the endoscope will cover all key site locations required for medical examination. For input data acquired during an endoscopy (examination), the relationship between each sample image in input data and a location of the sample image in motion track can be identified based on a labeling method.

For example, an experienced doctor may perform an endoscopy multiple times in order to obtain an image sequence of the correlated samples regarding motion tracks of multiple samples. For another example, multiple experienced doctors may perform one or more endoscopy examinations respectively to obtain more abundant training data. Having obtained sufficient training data, the motion model 410A can be trained based on the sequence of sample images and motion tracks of samples. Here, a endoscopic operation specification defines locations of all key sites to be examined, and an experienced doctor can ensure that the performed examination can meet the requirements in the specification to the greatest extent. By performing training using the training data obtained in this way, it can be ensured that the obtained motion model 410A can accurately reflect the relationship between the image and the motion track. In addition, the motion model 410A can also be obtained by means of computer simulation.

For convenience of description, an exemplary implementation according to the present disclosure will hereinafter be described with only an image sequence as an example of the input data 210. Processing is similar when the input data 210 is stored in other formats. For example, when the input data 210 is in a video format, an image sequence in the video can be acquired and the input data 210 can be processed for the image sequence

Hereinafter, a process obtaining the motion model 410A will be described with reference to FIG. 4B. FIG. 4B schematically shows a block diagram 400B of a process of acquiring the motion model 410A according to an exemplary implementation of the present disclosure. Training can be performed based on sample image sequences and sample motion tracks collected during historical examination. The multiple sample image sequences may be divided into multiple groups, each group comprising N>3 images. In turn, grouping of multi-frame sample images 410B (e.g., consecutive N-frame images from the T-Nth frame) may be input into a neural network layer 412B, and grouping of a multi-frame sample images 420B (e.g., consecutive N-frame images from the Tth frame) may be input to the neural network layer 412B. The grouping of images) is input to the neural network layer 422B; grouping of multi-frame sample images 430B (e.g., consecutive N-frame images from the T+Nth frame) may be input to a neural network layer 432B. In this way, the association between the image sequence and the motion track can be obtained.

It will be appreciated that only one implementation that may be used to obtain the motion model 410A is schematically shown above with reference to FIG. 4B. According to exemplary implementations of the present disclosure, the motion model 410A may be obtained according to other machine learning techniques currently known and/or to be developed in the future.

The motion track of the endoscope 210 may be determined based on the motion model 410A and the input data. According to an exemplary implementation of the present disclosure, the motion track of the endoscope 210 comprises a set of key site locations during operation of the endoscope 210. Here, the set of key site locations comprises at least part of a set of predetermined human body locations from the endoscope 210 during endoscopy examination, and the plurality of locations traversed during operation of the endoscope may be located around the key site locations within a predetermined range.

It will be appreciated that the set of key site locations here may be locations defined according to the endoscopic specification. For example, locations such as the pharynx, esophagus, cardia, pylorus, etc. may be comprised. Assuming that the endoscope passes through the pharynx and acquires 3 images at multiple locations near the pharynx during the operation (e.g., 0.5 cm anterior to the pharynx, pharynx, 0.5 cm after leaving the pharynx), it is possible to determine the motion track comprising the key site location “pharynx”. As the endoscope 210 moves further, the motion track may comprise more key site locations, e.g., pharynx, esophagus, and the like. The above locations can be further subdivided, for example, the upper part, the middle part and the lower part of the esophagus. In other words, the motion track here may comprise one or more key site locations through which the endoscope 210 moves.

According to an exemplary implementation of the present disclosure, the collected input data 230 may be input to the motion model 410A in a similar manner to the acquisition of the motion model 410A. For example, the input data 230 may be divided into a plurality of groups (each group comprises N-frame images), and the plurality of groups may be sequentially input into the motion model 410A. At this time, at a certain layer in the motion model 410A, a feature corresponding to the current N-frame images (as a latent variable) may be continuously output, and the feature may be iteratively input to a location of a next layer. The motion model 410A can output the motion track of the endoscope according to the input data.

According to an exemplary implementation of the present disclosure, using the motion model 410A, the input data can be mapped to a set of key site locations, respectively. Continuing to refer to FIG. 4B, as shown on the right side of FIG. 4B, CLSC(T) represents prediction of the location of the key site to which consecutive N-frame images starting from the T-th frame belong, and CLSN(T) represents prediction of the location of the key site to which consecutive N-frame images belong, and CLSP(T) represents prediction of the key site location to which the previous N frame images belong, and Y(T) represents prediction of the motion track to which the current image sequence belongs. The prediction of the motion track here may comprise multiple key site locations. For example, the prediction of the motion track may comprise: key site location 110->key site location 112->key site location 114; the prediction of the motion track may comprise: key site location 110->key site location 112->key site location 116. According to the current input N-frames images, the prediction of the motion track can comprise different key sites. The next destination location may be determined based on the prediction of the motion track. Further, information associated with other frames may be determined in a similar manner.

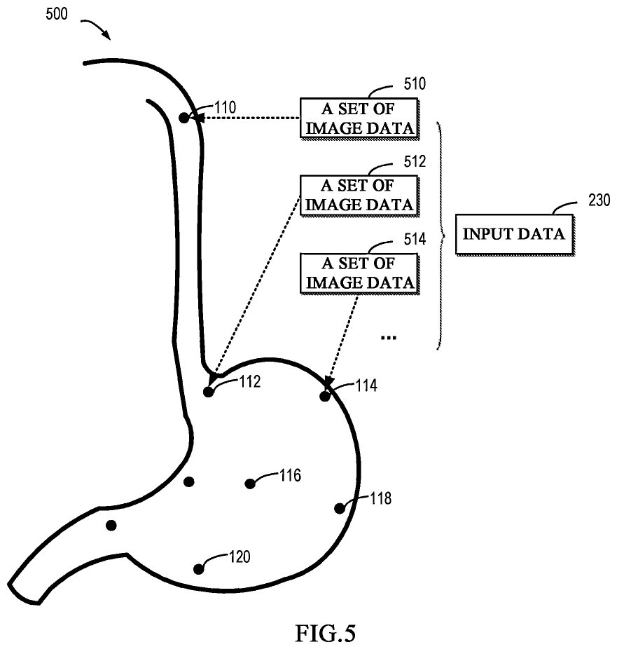

FIG. 5 schematically shows a block diagram 500 of a process of mapping input data to a set of key site locations according to an exemplary implementation of the present disclosure. As shown in FIG. 5, as the time the endoscope 210 moves within a body increases, the input data 210 will comprise more and more images. FIG. 5 only schematically shows an initial stage of endoscopy, and the endoscope 210 acquired a large number of images near key site locations 110, 112 and 114.

Using the method described above, images can be mapped to corresponding key site locations. For example, a set of image data 510 in an image sequence can be mapped to a key site location 110 to indicate that the set of image data 510 is collected near the key site location 110. Similarly, a set of image data 512 in an image sequence can be mapped to the key site location 112, and a set of image data 514 in an image sequence can be mapped to key site locations 114, and so on.

With exemplary implementations of the present disclosure, it is possible to determine locations where various image data was acquired based on input data 230 collected during operation of the endoscope 210. Compared with a technical solution that completely relies on the doctor's personal experience and judgment, the above-mentioned technical solution can determine the key site locations associated with image data in a more accurate manner, thereby helping to select which images to store later.

According to an exemplary implementation of the present disclosure, the motion track may be determined based on a time sequence in which image sequences associated with key site locations were collected. With continued reference to FIG. 5, it has been determined that the set of image data 510 is associated with the key site location 110, the set of image data 512 is associated with the key site location 112, and the set of image data 514 is associated with the key site location 114. It is assumed that the time sequence of collection of each image is: the set of image data 510, the set of image data 512, and the set of image data 514. At this time, it can be determined that a motion track 1 comprises: key site location 110->key site location 112->key site location 114.

It will be appreciated that the motion track comprises key site locations in time sequence. Thus, if the order of a set of key site locations is different, it represents different motion tracks. For example, a motion track 2 may comprise: key site location 110->key site location 114->key site location 112. Then the motion track 2 and the motion track 1 are different motion tracks.

In addition, the motion track may also be the actual motion track of the endoscope in the body part determined based on the input data. The actual motion track comprises both key site locations and non-key site locations, so as to reflect operation behaviors of the endoscope in real time, thereby better analyzing and assisting in guide of the inspection operation of the endoscope.

With the exemplary implementation of the present disclosure, the motion track of the endoscope 210 can be recorded in a more accurate manner based on the time sequence in which respective image data are collected. Further, the determined motion track can also be used for post-processing, for example, the key site location that should be reached can be determined based on the key site location that the endoscope 210 has reached.

Generally speaking, in the process of performing endoscopy, the doctor needs to manipulate the endoscope to reach the desired key site location on the one hand, and also need to store images for later diagnosis on the other hand. Since the image sequence collected during the examination process will occupy a large amount of storage space, usually the doctor only selects an appropriate angle based on his own experience to collect and store images after reaching near the key site. For example, a foot pedal may be provided at the endoscopic inspection device which the doctor may depress in order to store images. This can lead to situations where the doctor misses certain key site locations and/or where the stored images are of poor quality and cannot be used for diagnosis.

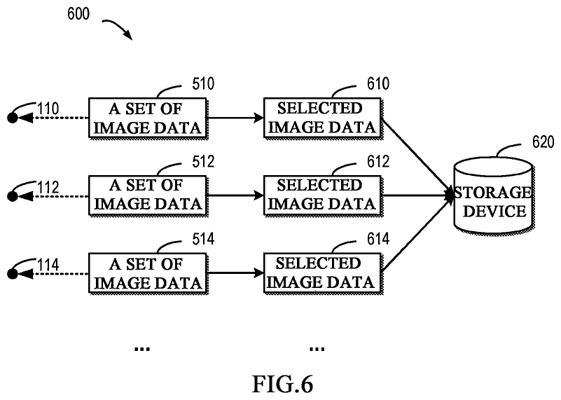

According to an exemplary implementation of the present disclosure, image analysis may also be performed on an already determined set of images in order to select therefrom an image that best reflects the state of the human body at a certain key site location. In the following, more details on selecting and storing images will be described with reference to FIG. 6. FIG. 6 schematically shows a block diagram 600 of a process for selecting images associated with key site locations for storage in accordance with an exemplary implementation of the present disclosure. Specifically, for a given key site location in a set of key site locations, a given set of images in the input data that are mapped to the given key site location can be determined.

As shown in FIG. 6, the image quality evaluation of a given set of images can be determined based on the image quality of a given set of image data. In turn, images for storage may be selected based on the determined image quality assessment. In FIG. 6, a set of image data 510 involving key site locations 110 has been determined, at which point an image quality evaluation can be determined for the set of image data 510. Then, the selected image data 610 may be obtained from the set of image data 510 and stored in a storage device 620 based on the image quality evaluation. Similarly, selected image data 612 can be retrieved from the set of image data 512 and stored in the storage device 620; and selected image data 614 can be retrieved from the set of image data 514 and stored in the storage device 620. In turn, information about the stored images can be displayed to the doctor, e.g., the number of images that have been stored, associated key site locations, and the like.

It will be appreciated that image quality here can have many meanings. For example: an image that can better reflect the location of the key sites to be checked. For example, the image quality may comprise one or more of the following: the clarity of the human mucosa in the image collected by the endoscope, whether the mucosa is contaminated, whether the mucosa is covered by secretions, etc., the shooting angle of the endoscope, etc. If the human mucous membranes are clearly visible, uncontaminated, and not obscured by secretions, the image can be determined to be of high quality. On the contrary, it can be determined that the image has lower quality.

It will be appreciated that image quality may be determined in a number of ways here. For example, sharpness of the image can be determined based on the method of image processing so as to obtain the image quality evaluation. For another example, a quality prediction model can be established by using pre-labeled sample data based on machine learning. According to exemplary implementations of the present disclosure, other image processing techniques that have been developed and/or will be developed in the future may also be employed to obtain image quality evaluation.

With exemplary implementations of the present disclosure, one or more images with the best image quality can be selected from a large number of images acquired at a given key site location. Compared with a technical solution of manually selecting and storing images based on the personal experience of doctors, efficiency of selecting images can be significantly improved, the time taken by doctors to select and store images can be shortened, and the efficiency of endoscopy can be improved. On the other hand, since the mapping, selection and storage of images are carried out in an automatic manner, omissions due to doctor error can also be avoided as much as possible. In addition, further, an image with better image quality can be selected according to the time sequence of the acquired input data (e.g., the image sequence or the relationship between images at key site locations).

According to an exemplary implementation of the present disclosure, motion track evaluation may be determined based on the motion track of the endoscope 210 and are determined motion track of the endoscopy. The predetermined motion track here may be a sequence of a series of key site locations defined according to the endoscope operation specification. For example, the predetermined motion track may comprise pharynx->esophagus->cardia->pylorus and the like. It is expected that the doctor can operate motion of the endoscope according to the predetermined motion track, so the evaluation can be determined based on consistency of an actual motion track of the endoscope 210 with the predetermined motion track.

According to exemplary implementations of the present disclosure, evaluation may comprise various types. For example, a score can be expressed as evaluation in a range (such as a real number between 0-1); a score can be expressed as evaluation (such as high, medium, low); literal depiction can be expressed as evaluation; or an image or other ways can be expressed as evaluation.

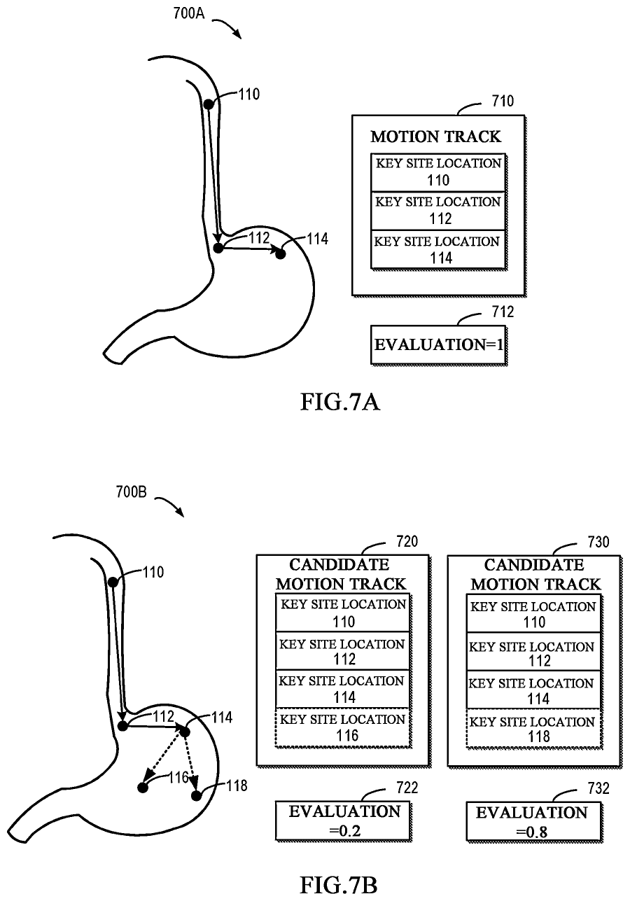

Hereinafter, more details on determining the motion track evaluation will be described with reference to FIG. 7A. FIG. 7A schematically shows a block diagram 700A of a data structure of a motion track according to an exemplary implementation of the present disclosure. In FIG. 7A, a motion track 710 of the endoscope 210 comprises three key site locations: key site locations 110, 112 and 114. At this point, the endoscope 210 is located at the key site location 114, and the motion track evaluation 710 may be determined based on comparing the motion track 710 with the predetermined motion track of the endoscopy. Further, relevant evaluation can be displayed to the doctor.

It will be appreciated that the evaluation may be determined in a number of ways here. A numerical range for the evaluation can be specified, for example, a scale of 0-1 can be expressed as the evaluation. It is assumed that the predetermined motion track comprises: key site location 110->key site location 112->key site location 114->key site location 118 . . . , and the motion track 710 at this time comprises key site location 110->key site location 112->key site locations 114. It may be determined that the motion track 710 completely matches a beginning of the predetermined motion track, and thus higher evaluation 712 may be given to the motion track 710, e.g., the evaluation 712 may be set to a maximum score of 1. For another example, if the predetermined motion track deviates, the numerical value of the evaluation can be reduced at this time, for example, the evaluation can be set to 0.8.

It will be appreciated that the principles for determining the evaluation have been described above by way of illustration only. According to an exemplary implementation of the present disclosure, an evaluation prediction model can be established by using pre-labeled sample data based on machine learning. According to exemplary implementations of the present disclosure, other prediction techniques that have been developed and/or will be developed in the future may also be employed to obtain an evaluation of the motion track.

Hereinafter, more details regarding the determination of the next destination location will be described with reference to FIG. 7B. According to an exemplary implementation of the present disclosure, a set of candidate locations may be determined based on one or more key site locations near the last key site location in the motion track. FIG. 7B schematically shows a block diagram 700B of a process of providing a next destination location according to an exemplary implementation of the present disclosure. As shown in FIG. 7B, a set of candidate locations for the endoscope 210 at a next time point may be determined first. Continuing the above example, the endoscope 210 is currently located at the key site location 114 and the key site locations 116 and 118 near the key site location 114. At this point, the set of candidate locations may comprise the key site locations 116 and 118. In turn, evaluation of each candidate location in the set of candidate locations may be determined, and the next destination location selected from the set of candidate locations based on the determined evaluation.

Specifically, for a given candidate location in the set of candidate locations, a candidate motion track of the endoscope 210 may be generated based on the motion track and the candidate location. As shown in FIG. 7B, based on the motion track 710 and the key site locations 116, a candidate motion track 720 may be generated; based on the motion track 710 and the key site locations 118, a motion candidate motion track 730 may be generated. Then, the evaluation 722 and 732 of the two candidate motion tracks 720, 730 may be determined based on the candidate motion tracks 720, 730 and the predetermined motion tracks of the endoscopy, respectively, using the method described above. As shown in FIG. 7B, since the evaluation 732 is higher than the evaluation 722, higher evaluation can be given to the key site location 118, and the key site location 118 can be used as the next destination location.

With the exemplary implementation of the present disclosure, a key site location that best matches the predetermined motion track of the endoscope 210 can be preferentially recommended to the doctor as the next destination location for moving the endoscope 210. In this way, guidance can be given for motion operation of the doctor, which can reduce potential risk of missing key site locations while improving efficiency of the endoscopy. Further, since the motion of the endoscope in the human body may cause discomfort to the patient, improving inspection efficiency can shorten the length of time for the endoscope inspection, thereby reducing pain of the patient.

It will be appreciated that although specific examples of providing the next destination location are described above with reference to the accompanying drawings. According to an exemplary implementation of the present disclosure, a subsequent recommended path comprising one or more key site locations may also be provided. The doctor can move the endoscope along the recommended path to cover all key sites required for the endoscopy.

According to an exemplary implementation of the present disclosure, the candidate motion track of the endoscope may also be directly generated based on the motion model 410A and the input data. It will be appreciated that the motion model 410A may be built on an end-to-end basis during training. Herein, input of the motion model 410A may be designated as the image sequence, and output of the motion model 410A may be designated as the candidate motion track. Here, the candidate motion track may comprise a set of key site locations corresponding to the input image sequence and the next candidate key site location. When using the motion model 410A, a set of image sequences currently collected by the endoscope may be input to the motion model 410A in order to obtain a candidate motion track. At this time, the doctor can operate the endoscope to move along the candidate motion track in order to cover all key site locations.

According to an exemplary implementation of the present disclosure, by using a labeled historical sample image sequence and a historical sample candidate motion track, the motion model 410A comprising an association relationship between the image sequence and the candidate motion track can be directly obtained. With exemplary implementations of the present disclosure, a training process can be performed and corresponding models obtained directly based on historical sample data. In this way, the operation process can be simplified so as to improve efficiency of obtaining candidate motion track.

According to an exemplary implementation of the present disclosure, further, information related to the operation behavior of the endoscope may be transmitted and/or stored.

According to an exemplary implementation of the present disclosure, information related to current doctor's operations can be output in real-time, and statistical and analytical function are provided accordingly. For example, the method 300 described above may further provide the following function: determine duration of the endoscopy, determine information on key site locations scanned, determine information on key site locations not scanned, determine the next destination location information, determine operational evaluation of the endoscopy by the doctor, determine whether images collected about various key sites locations are qualified, and so on.

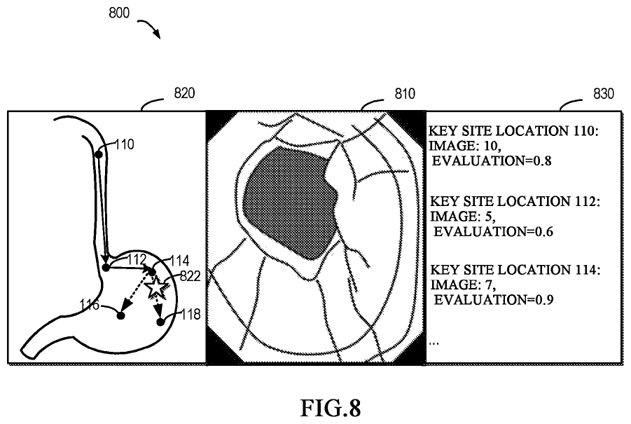

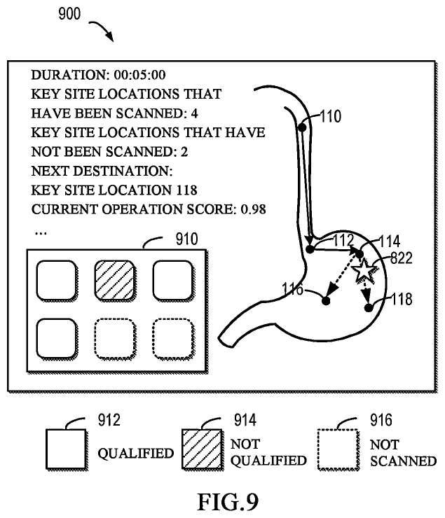

Hereinafter, a related function of outputting information related to an operation behavior will be described with reference to FIGS. 8 and 9. According to an exemplary implementation of the present disclosure, the function of outputting the above-mentioned information can be combined with an existing endoscope display interface. FIG. 8 schematically shows a block diagram of a user interface 800 providing medical assistance operations in accordance with an exemplary implementation of the present disclosure. As shown in FIG. 8, the user interface 800 may comprise: an image display part 810 for displaying the video 220 collected by the endoscope 210 in real time; a motion track management part 820 for displaying the trajectory motion that the endoscope 210 passed through and a hint of a next destination location; and a statistics section 830 for displaying information about images collected during endoscopy.

As shown in the motion track management part 820, the solid line indicates that the motion track that the endoscope 210 passed is: key site location 110->key site location 112->key site location 114. The dashed part represents a trajectory from the current location of the endoscope 210 (the key site location 114 to the next destination location (i.e., the key site locations 116 and 118). The next destination location may be set as the key site location 118 based on the method described above with reference to FIG. 7B. Further, a star mark 822 can be used to indicate that a recommended next destination location is the key site location 118. At this point, the doctor can move the endoscope 210 to the key site location 118 at the next time point.

As shown in a statistics section 830, relevant information about the collected images may be displayed. For example, for the key site location 110, 10 images are selected and overall score for the 10 images is 0.8. It will be appreciated that an upper limit on the number of images expected to be acquired for each key site may be predefined, e.g., the upper limit may be defined as 10. The 10 images shown here may be images with higher image quality selected according to the method described above with reference to FIG. 6, and the evaluation 0.8 here may be comprehensive evaluation obtained based on individual image quality evaluation.

According to an exemplary implementation of the present disclosure, a lower limit on image quality evaluation may also be set. For example, it can be set to select only images with a score higher than 0.6. According to an exemplary implementation of the present disclosure, which images are desired to be stored may also be selected based on both the upper limit of the number of images and the lower limit of the image quality evaluation. The statistics section 830 further shows statistics about other key site locations: for the key site location 112, 5 images are selected and the overall score for the 5 images is 0.6; and for the key site location 114, 7 images are selected, and the overall evaluation of the 7 images is 0.9.

According to an exemplary implementation of the present disclosure, the user interface for managing the motion of the endoscope 210 may be separated from the existing endoscope display interface. FIG. 9 schematically shows a block diagram of another user interface 900 providing medical assistance operations in accordance with an exemplary implementation of the present disclosure. As shown in FIG. 9, relevant information about medical assistance operations may be displayed in the separate user interface 900. In the user interface 900, information related to the operation behavior can be output.

According to an exemplary implementation of the present disclosure, information about the image selected for key site locations may also be displayed in an area 910. For example, the area 910 may comprise thumbnails of images. Assuming that the endoscopy procedure requires collecting images of 6 key site locations, images of 4 key site locations are collected and images of the remaining 2 key site locations are not collected. Legends 912, 914, and 916 may be employed to represent images of different types of key site locations, respectively. For example, the legend 912 indicates that a qualified image is collected at a key site location, and the legend 914 indicates that a qualified image is not collected at a key site location, and the legend 916 indicates that a key site location is not scanned. With the exemplary implementation of the present disclosure, the key sites locations scanned and not scanned, and those at which images are not qualified can be displayed to the doctor in a visual manner, thereby facilitating the doctor's follow-up operation.

According to an exemplary implementation of the present disclosure, after an image is selected for storage, image anomalies associated with a given key site location may be identified based on the selected image. Further, the identified image anomalies can be displayed. Specifically, content of the image may be analyzed based on image recognition technologies currently known and/or to be developed in the future in order to determine possible image anomalies at the location of the key site. For example, image anomalies can be indicative of ulcers, tumors, and the like. With the exemplary implementation of the present disclosure, images that may indicate anomalies can be identified, thereby assisting a doctor's diagnosis.

According to an exemplary implementation of the present disclosure, based on the input data, a working state of the endoscope 210 is identified. It will be appreciated that a variety of operational states may be involved during operation of the endoscope 210. For example, during activation of the endoscope 210 and insertion of the endoscope 210 into a patient, image content collected by the endoscope 210 will be different. The patient being examined can be determined based on analysis of images collected by the endoscope 210, and the endoscope can be determined whether the endoscope is currently inside or outside the patient, and a current examination site can be determined (e.g., stomach or bowel, etc.). For example, if part of an image sequence relate to in vitro images and a subsequent part of the images are converted to in vivo images, it may be determined that an in vitro/in vivo switch occurs. Further, switch of the working states can be recognized.

As another example, between examinations for two patients, a patient switch may be determined to occur based on analysis of input data collected by the endoscope 210. Specifically, a patient switch may be determined to occur when the image sequence comprises an in-vivo image, an in-vitro image, and then an in-vivo image different from the previous examination. As another example, endoscopy may involve different body parts. At this time, an examination location switch] may be determined based on analysis of the image acquired by the endoscope 210. Specifically, switch of inspection locations such as esophagoscope, gastroscope, duodenum, and colonoscope can be determined. With exemplary implementations of the present disclosure, relevant configurations for medical assistance operation may be selected based on detected switch. For example, corresponding motion models can be selected for gastroscopes and colonoscopes.

It will be appreciated that the endoscopy requires insertion of the endoscope 210 into a patient, and preparatory work needs to be performed prior to examination. According to an exemplary implementation of the present disclosure, a preparation status of a person performing an endoscopy may be identified based on input data. The preparation status here describes how qualified the person's physical state is for performing endoscopy. For the patient, preparations such as abstaining from eating and drinking, emptying the digestive tract, taking medication as prescribed to empty and clean the digestive tract, etc. For the doctor, preparations such as rinsing the stomach, insufflation of the stomach to examine folds, etc.

Specifically, if collected gastroscopic images comprise food residues, etc., it can be determined that the patient's preparation state is poor and requirements for emptying the digestive tract are not met. If the collected gastroscopic image comprises a large amount of secretions, etc., it can be determined that the doctor's cleaning operation is insufficient, and the doctor should be prompted to perform further cleaning operation. Further, readiness of recognition can be output. The output can be in the form of displays or other prompts. With exemplary implementation of the present disclosure, the patient and the doctor may be prompted for corresponding precautions based on the preparation state, respectively.

It will be appreciated that although specific examples of determining a preparation state based on images in the input data 230 are described above, according to exemplary implementations of the present disclosure, the preparation state may also be determined based on a dedicated sensor deployed at the endoscope (e.g., a sensor monitoring in vivo environmental parameters).

During the motion of the endoscope 210 in the human body, if the motion is too fast, key site locations will be missed, and the patient may also be uncomfortable such as nausea and pain. Therefore, it is also expected that a motion state of the endoscope 210 can be monitored based on smoothness of the motion, so that the motion track of the endoscope can cover all key site locations and reduce discomfort of the patient. According to an exemplary implementation of the present disclosure, the smoothness of the motion of the endoscope 210 may be identified based on a set of time points when the endoscope 210 reaches a set of key site locations. The smoothness here may refer to how smooth the motion of the endoscope 210 in the patient is. Further, the smoothness recognized can be displayed.

According to an exemplary implementation of the present disclosure, speed evaluation of the motion speed of the endoscope 210 may be determined based on the smoothness. For example, if the endoscope 210 moves a larger distance in a shorter period of time, it indicates that the motion of the endoscope 210 is more violent and should be avoided. At this point, lower speed evaluation can be given, and the doctor can be prompted to slow down from vigorous motion in order to prevent missing key site locations.

As another example, if the motion of the endoscope 210 is moderate, higher speed evaluation may be given. As another example, if the endoscope 210 moves only a small distance over a longer period, the overall time of the endoscopy will increase despite of smoother motion, thus lowering the speed evaluation and prompting the doctor to move the endoscope as soon as possible to the next destination location. For another example, it is also possible to monitor velocity distribution during the endoscopy, assuming that the endoscope 210 stays near 5 key sites locations in the first half of the entire inspection, and quickly passes the remaining 33 key sites locations in the second half, the second half of the inspection is likely to be insufficient, and lower speed evaluation is given at this time.

With respect to a technical solution of determining whether the doctor's operation is sufficient based on whether the overall examination time reaches a desired time (e.g., 10 minutes), with an exemplary implementation of the present disclosure, whether the doctors' operation meets the predetermined standard may be determined based on velocity distribution of the endoscope 210. It will be appreciated that although specific examples of determining the smoothness based on images in the input data 230 are described above, the smoothness may also be determined based on a velocity sensor deployed at the endoscope, according to exemplary implementations of the present disclosure.

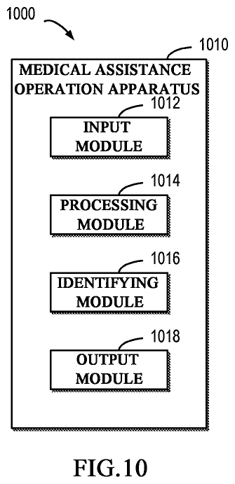

The details of a medical assistance operation method have been described above with reference to FIGS. 2 to 9. Hereinafter, each module in medical assistance operation apparatus will be described with reference to FIG. 10. FIG. 10 schematically shows a block diagram 1000 of medical assistance operation apparatus 1010 (or a medical assistance information processing device 1010) according to an exemplary implementation of the present disclosure. As shown in FIG. 10, the medical assistance operation apparatus 1010 is provided, comprising: an input module 1012 configured to obtain input data from an endoscope; and an output module 1018 configured to output information related to an operation behavior of the endoscope and determined based on input data.

According to an exemplary implementation of the present disclosure, the input data comprises image data collected at multiple locations during operation of the endoscope.

According to an exemplary implementation of the present disclosure, the device 1010 further comprises a processing module 1014 configured to determine the information related to the operation behavior of the endoscope based on the input data.

According to an exemplary implementation of the present disclosure, the processing module 1014 is further configured to determine a next destination location of the endoscope based on the input data.

According to an exemplary implementation of the present disclosure, the processing module 1014 is further configured to: determine the motion track of the endoscope based on the input data.

According to an exemplary implementation of the present disclosure, the motion track is represented by a predetermined set of key site locations.

According to an exemplary implementation of the present disclosure, the processing module 1014 is further configured to determine a next destination location of the endoscope based on the motion track.

According to an exemplary implementation of the present disclosure, the processing module 1014 is further configured to: determine a set of candidate locations of the endoscope at the next point in time; determine evaluation of each candidate location in the set of candidate locations; and select the next destination location from the set of candidate locations based on the determined evaluation.

According to an exemplary implementation of the present disclosure, the processing module 1014 is further configured to: generate a candidate motion track of the endoscope for a given candidate location in the set of candidate locations, based on the motion track and the given candidate location; and determine evaluation of the candidate location based on the candidate motion track and a predetermined motion track of the endoscopy.

According to an exemplary implementation of the present disclosure, the processing module 1014 is further configured to: determine the evaluation of the motion track.

According to an exemplary implementation of the present disclosure, the apparatus 1010 further comprises an identification module 1016 configured to: identify a working state of the endoscope, the working state comprising at least any one of the following: patient identification, in vitro and in vivo situation, and examination parts.

According to an exemplary implementation of the present disclosure, the identification module 1016 is further configured to: identify the switch of the working state.

According to an exemplary implementation of the present disclosure, the device 1010 further comprises an identification module 1016 configured to: identify a readiness state of a part for endoscopy based on the input data, the readiness state indicating qualification of an examination part for the endoscopy.

According to an exemplary implementation of the present disclosure, the apparatus 1010 further comprises an identification module 1016 configured to: determine the smoothness of the motion of the endoscope based on a set of time points when the endoscope reaches a set of key site locations.

According to an exemplary implementation of the present disclosure, the processing module 1014 is further configured to: obtain a set of key site locations based on the input data; and determine the motion track based on the temporal order of the input data associated with the key site locations.

According to an exemplary implementation of the present disclosure, the processing module 1014 is further configured to: determine a set of image data mapped to key site locations in the input data, determine image quality assessment of a set of image data based on image quality of the set of image data; and select image data of the set of image data for storage based on the determined image quality assessment.

According to an exemplary implementation of the present disclosure, the apparatus 1010 further comprises an identification module 1016 configured to: identify image anomalies at key site locations based on the selected image data.

According to an exemplary implementation of the present disclosure, the processing module 1014 is further configured to: obtain a first model describing the endoscopy, the first model comprising relationship between sample input data collected at multiple sample locations during the endoscopy and sample motion track of the endoscope used to collect the sample input data

According to an exemplary implementation of the present disclosure, the processing module 1014 is further configured to: obtain sample input data collected in an endoscopy performed in accordance with an endoscopic operational specification; obtain a sample motion track associated with the sample input data; and train a first model based on the sample input data and the sample motion track.

According to an exemplary implementation of the present disclosure, the processing module 1014 is further configured to: obtain a second model describing the endoscopy, the second model comprising relationship between sample input data collected at multiple sample locations during the endoscopy and corresponding key site locations for multiple locations where sample input data was collected; and determine the motion track of the endoscope based on the second model, the input data, and the collection time of the image data.

According to an exemplary implementation of the present disclosure, determining information related to the operation behavior of the endoscope based on the input data comprises determining at least any one of: a current location of the endoscope; image data collected at the current location; the motion track of the endoscope; the next destination location of the endoscope; the statistics of the input data; and the statistics of the operation behavior.

According to an exemplary implementation of the present disclosure, the input data comprises at least any one of: video data; a set of image sequences arranged in time sequence;

and a plurality of image data with time information.

According to an exemplary implementation of the present disclosure, the output module 1018 is further configured to: transmit information related to the operation behavior of the endoscope.

According to an exemplary implementation of the present disclosure, each module of the medical assistance operation apparatus 1010 may be implemented by one or more processing circuits.

As mentioned above, with the further development of medicine, after doctors perform various medical tests such as endoscopy, it is expected to perform quality control on the operation behaviors associated with the tests. When performing quality control, information related to operation behaviors of the endoscope described in the foregoing embodiments can be used. The purpose of quality control may comprise displaying information related to the operation behaviors of the endoscope to operators such as doctor operators using medical testing device and potentially to department leaders, or hospital leaders, etc. so as to show quality of results of the operation behaviors, deviations from recommended operation, suggested directions for modification, and any possible statistical information, and in turn, helping doctors improve their operation on medical testing devices.

In the following embodiments, information associated with the operation behaviors of the endoscope may also be referred to as first information associated with operation behaviors of the medical testing device. According to some exemplary implementations of the present disclosure, the first information may also be referred to as displayed information, and may also be referred to as entered information, or referred to as cloud storage information. The first information may be data entered into a cloud server or a cloud memory.

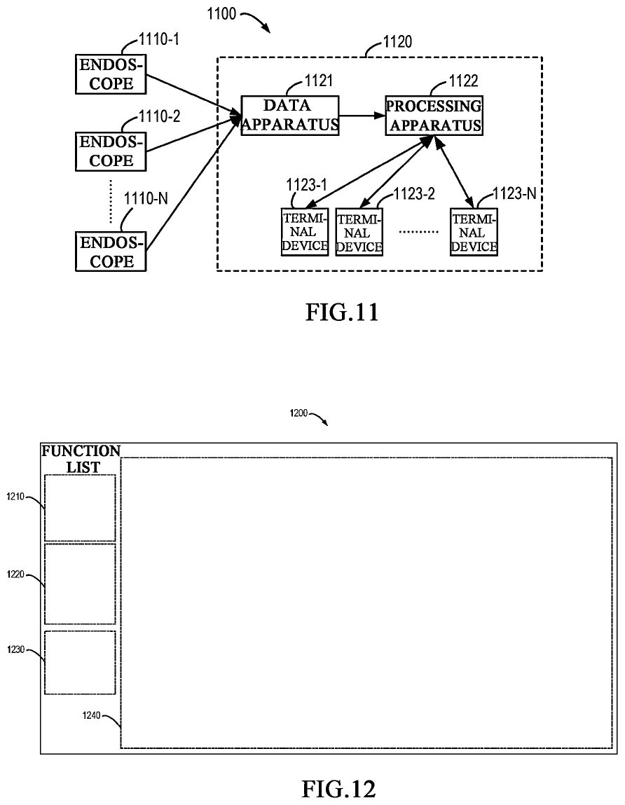

FIG. 11 schematically shows a schematic diagram of a quality control environment 1100 that may be used to implement exemplary implementations of the present disclosure.

As shown in FIG. 11, the quality control environment 1100 comprises a plurality of endoscopes 1110-1, 1110-2 . . . 1110-N, which may be collectively referred to as the endoscope 1110, and a quality control system 1120. The quality control system 1120 comprises a data device 1121, a processing device 1122 comprising a cloud storage device, and a plurality of terminal devices 1123-1, 1123-2 . . . 1123-N collectively referred to as a terminal device 1123.

It should be understood that the quality control environment 1100 is merely exemplary and not limiting, and that it is expandable to comprise more endoscopes, data devices, processing devices, and terminal equipment to meet requirements from more users to conduct quality control of medical examination operation at the same time.

The data device 1121 in FIG. 11 may be implemented as the medical assistance operation device 1010 (or as the medical assistance information processing device 1010) described according to FIG. 10. According to some exemplary implementations of the present disclosure, the data device 1121 may comprise a local central processing unit or a graphics processing unit, which may receive data collected by the endoscope 1110 as the input data by means of a data acquisition card, wired transmission, and wireless transmission, etc. After receiving the input data from the endoscope 1110, the data device 1121 may analyze and process the input data in accordance with the details of the medical assistance operation apparatus already described above with reference to FIGS. 2 to 9 to generate the first information in a correlation with the operation behaviors of the medical testing device then provided to the processing device 1122 by means of a data acquisition card, wired transmission, wireless transmission, etc.

The processing device 1122 may perform data interaction with the terminal device 1123, comprising receiving a request from the terminal device 1123 for the first information associated with the operation behaviors of the medical testing device, and providing the terminal device 1123 with the first information the first information associated with the operation behavior of the medical testing device in response to the aforementioned request.

According to other exemplary implementations of the present disclosure, the processing device 1122 may comprise the cloud server connected to the processing device 1122 through wired or wireless communication, and the processing device 1122 receives data from the data device 1121 and calculates the received data to obtain data results. The data results are stored on the cloud server. The terminal device 1123 is connected to the cloud server through wired or wireless communication, so as to obtain the data result calculated by the cloud server.

In addition, according to other exemplary implementations of the present disclosure, the terminal device 1123 may also directly perform data interaction with the data device 1121, and at this time the data device 1121 may have function of the processing device 1122, or at least partially act as the processing device 1122. According to some exemplary implementations of the present disclosure, the terminal device 1123 may be any existing or possibly developed terminal device, such as a desktop computer, a laptop computer, and a mobile phone. The medical quality control involved in the exemplary implementation of the present disclosure is implemented through data interaction between the processing device 1122 and the terminal device 1123.

According to some exemplary implementations of the present disclosure, the quality control system may comprise a background management system and a client system, wherein the background management system may be implemented as a gastrointestinal endoscopy quality control background management system, and the client system may be implemented WeChat applet or other applications for quality control of gastroscopy. The background management system may be installed in the processing device 1122, installed in the cloud server of the processing device 1122, or installed in the terminal device 1123 together with the client system. The client system may be installed in the terminal device 1123, installed in the processing device 1122, or installed in the cloud server of the processing device 1122. The present disclosure does not limit specific installation locations of the background management system and the client system.

FIG. 12 shows a schematic diagram 1200 when a background management system is running. The background management system shown in the schematic diagram 1200 comprises four functional parts 1210, 1220, 1230 and 1240, which may respectively correspond to different functional modules of the background management system.

The function part 1210 shows the system management function provided by the background management system, comprising account management and role management, which can be used for operations such as background data management account configuration and permission setting by a super administrator or an advanced user. The function part 1220 shows hospital management function provided by the background management system, comprising hospital management, doctor management, and department management, which can be used for operations such as hospital management, doctor authority assignment, and department management, for example, performed by the super administrator or the advanced user. The function part 1230 shows the device management function provided by the background management system, which can be used for, for example, operations such as terminal device location setting, authority assignment, and query log-in history performed by the super administrator or the advanced user. The function part 1240 shows the displayed content provided by the background management system, comprising account information, where the account information may comprise serial number, account number, user name, the hospital, creation time, activation status, update time, etc., and the super administrator or the advanced user can use the corresponding icon after each piece of account information on the function part 1240 to enter an account editing interface to realize account editing function, or delete the corresponding account.

It should be understood that the schematic diagram 1200 shows, for example, a schematic diagram after the super administrator or the advanced user logs in to the background management system. According to some exemplary implementations of the present disclosure, the super administrator or the advanced user may be a department manager or a hospital manager, where the department manager may comprise the department director and the hospital manager may comprise a dean. Users with different management levels have different management rights.

Operations associated with the schematic diagram 1200 may comprise that the user uses the background management system to interact with the functional parts 1210, 1220, 1230 and 1240 by means of input or touch, thereby entering specific functional modules or calling specific functional modules to display or output information.

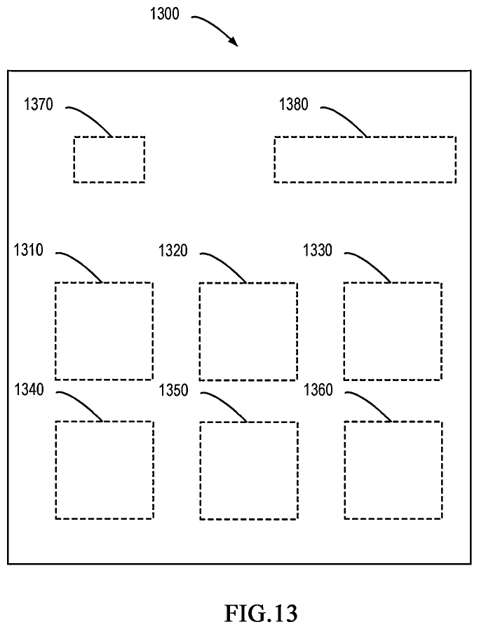

FIG. 13 shows a schematic diagram 1300 when a client system is running. The schematic diagram 1300 shows six functional parts 1310, 1320, 1330, 1340, 1350, 1360, 1370, and 1380, which may correspond to different functional modules of the client system, respectively. According to some exemplary implementations of the present disclosure, the function part 1310 corresponds to a current check-in function module; the function part 1320 corresponds to a supplementary check-in record function module, the function part 1330 corresponds to the department inspection record function module; the function part 1340 corresponds to a My inspection record function module; the function part 1350 corresponds to a My quality control analysis function module; the function part 1360 corresponds to a department quality control analysis function module; the function part 1370 corresponds to a functional module displaying information such as pictures and names representing logged-in users; the functional section 1380 corresponds to a functional module that displays information indicating successful login on a graphical user interface. The client system may be used, for example, by a doctor or nurse as an operator or a user of the medical device, and the operator and the user are collectively referred to below as the operator. It should be understood that the schematic diagram 1300 shows, for example, a schematic diagram after an operator logs into the client system.

According to some exemplary implementations of the present disclosure, an interaction process between the client system and the user comprises receiving a user login instruction, and responding to the login instruction for displaying various functional parts.

Operations associated with the schematic diagram 1300 may comprise that the user interacts with the functional parts 1310, 1320, 1330, 1340, 1350, 1360, 1370 and 1380 by means of client system input or touch, so as to enter specific functional modules or call specific function modules to display or output information.

According to some exemplary implementations of the present disclosure, the user of the client system can use user identification information to log in to the client system, and the user identification information for logging in can comprise phone number, Wechat ID, doctor's certificate number, nurse certificate number, etc., and associated verification information, such as passwords, fingerprints, face recognition, pupil recognition, etc. If the user logs in successfully, the client system may display information indicating successful login on the graphical user interface, e.g., corresponding to function section 1380, and may display information such as the picture and the name representing the logged-in user, e.g., corresponding to function section 1370. At the same time, the user identification information can be saved in the terminal device, so that the user does not need to input the user identification information again in a subsequent login. In addition, the client system can also maintain a logged-in state of the user, so that the user can use the client system in a logged-in state at any time without actively performing a log-out operation. If the user fails to log in, the client system does not display information such as the picture and the name of the user on the graphical user interface, but may display information indicating a failed login.

According to some exemplary implementations of the present disclosure, the client system may display different function modules for different logged-in users. For example, when the logged-in user is a general doctor or a general nurse, the client system only displays the functional parts 1310, 1320, 1340, 1350, 1370 and 1380 in the graphical user interface. The functional parts 1330 and 1360 are displayed only when the logged-in user is a department manager or a hospital manager with higher authority, for example.

According to other exemplary implementations of the present disclosure, the client system may always display all functional parts and make only some functional parts available for different logged-in users. For example, when the logged-in user is the general doctor or the general nurse, the client system may make the displayed functional parts 1330 and 1360 unavailable, e.g., not clickable.

It should be understood that the various information shown in the schematic diagram 1300 is only an example, and is not intended to limit the scope of protection of the present disclosure. Such information can be adjusted in name and type according to needs, to be displayed or not to be displayed, or to change the displayed location and form, without affecting the normal implementation of the embodiments of the present disclosure.

The functional section 1310 is used for the operator to check in, e.g., to associate the medical device to be used with the client system. When the operator selects the function part 1310 by, for example, touching, the check-in function of the client system is entered.

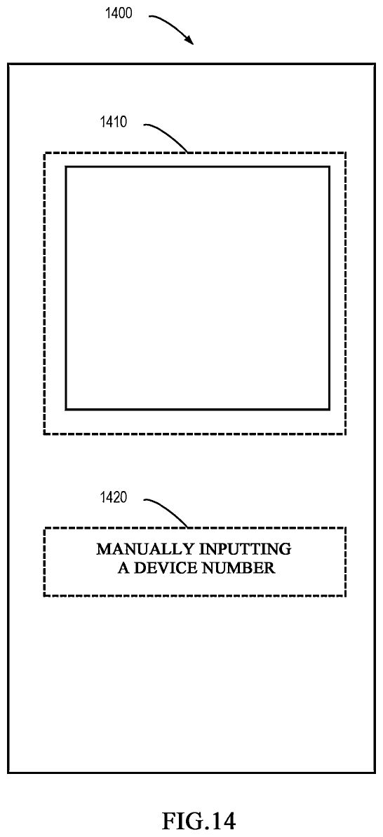

FIG. 14 shows a schematic diagram 1400 when a client system is running. The diagram 1400 shows the check-in functionality of a client system. The schematic diagram 1400 shows the two functional parts 1410 and 1420, which may respectively correspond to different functional modules of the client system.

The functional part 1410 may support code scanning function such as scanning a two-dimensional code or a barcode, wherein the two-dimensional code or the barcode may correspond to a specific medical device. The function part 1420 may support input function such as manually inputting a device number of the medical device. After the operator uses the function part 1410 or 1420 to determine the medical device, the client system can complete association of the medical device with a user logging in to the client system.

Operations associated with the schematic diagram 1400 may comprise that a user interacts with the functional parts 1410 and 1420 by means of client system input or touch, thereby entering a specific functional module or calling a specific functional module to display or output information.

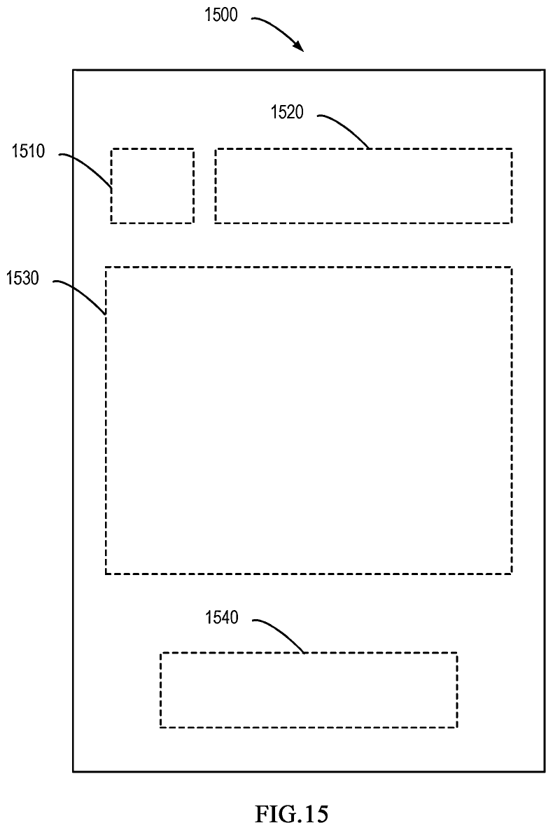

FIG. 15 shows a schematic diagram 1500 when a client system is running. The diagram 1500 shows a diagram after the client system completes associating a medical device with a user logged into the client system. Four functional parts 1510, 1520, 1530, and 1540 are shown in the schematic diagram 1500, which may correspond to different functional modules of the client system, respectively.

The function section 1510 corresponds to a function module that displays whether the medical device is performing examination. The function part 1520 corresponds to a function module that displays a user currently logged into the client system, the medical device number, and a corresponding department. The function part 1530 corresponds to a function module that displays examination operation performed on the user using the medical device. The function part 1540 corresponds to a function module for performing sign-out operation, the user of the client system disassociating of the medical device from the operator by a sign-out function part.

Operation associated with the schematic diagram 1400 may comprise that the user interacts with the functional parts 1510, 1520, 1530, and 1540 by means of client system input or touch, thereby entering a specific functional module or calling a specific functional module to display or output information.

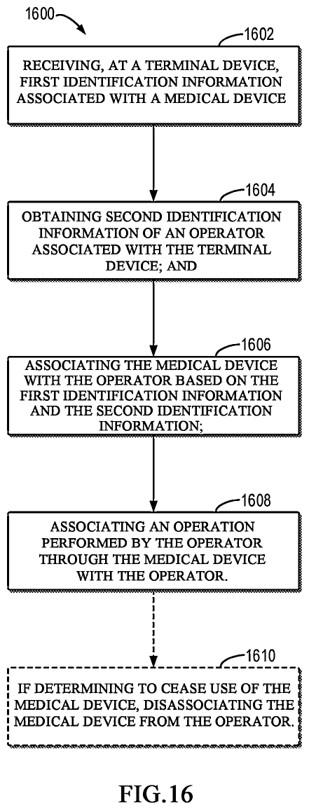

FIG. 16 schematically shows a flowchart of an information processing method 1600 according to an exemplary implementation of the present disclosure. The method 1600 may embody corresponding function of the client systems shown in FIG. 14 and FIG. 15, but may alternatively or additionally comprise other function. The method 1600 may also comprise additional steps not shown and/or steps shown may be omitted, as the scope of the present disclosure is not limited in this regard.

At block 1602, the terminal device receives first instruction information indicating at least one operation performed by a medical device from the user. According to some exemplary implementation manners of the present disclosure, a client system may be used on the terminal device to implement corresponding function. As mentioned above, the operator can use the terminal device to input the first identification information associated with a medical device into the terminal device, so that the terminal device can definitely identify a specific medical device.

According to some exemplary implementations of the present disclosure, receiving the first identification information may comprise receiving identification information in at least one of the following ways: scanning a two-dimensional code corresponding to the identification information; scanning a barcode corresponding to the identification information receiving an input of a device identification corresponding to the identification information; receiving an audio input corresponding to the identification information; receiving a video input corresponding to the identification information; and receiving a tactile input corresponding to the identification information. Through the above manner, the terminal device can definitely identify the specific medical device.

At block 1604, receiving second indication information indicating an operator of at least one operation from the user. The second identification information is used to specifically identify a specific user. According to some exemplary implementation manners of the present disclosure, the mobile terminal may determine that a user who has logged into the client system is an operator associated with the terminal device, and login information at this time is second identification information. According to other exemplary implementations of the present disclosure, the user who has logged into the client system may input another user's identification information through the client system, so that the terminal device determines the other user as the operator associated with the terminal device.

According to some exemplary implementations of the present disclosure, the second identification information may comprise at least one of the following: the operator's name, the operator's username, the operator's phone number, an image associated with the operator, and job title of the operator.

At block 1606, the terminal device associates the medical device with the operator based on the first identification information and the second identification information. According to some exemplary implementations of the present disclosure, since the operator has not used the medical equipment to operate at this time, the terminal device only associates the medical equipment with the operator. According to some other exemplary implementations of the present disclosure, at this time, the mobile terminal can associate the operation performed by the medical device with the operator

At block 1608, the terminal device associates operations performed by the operator through the medical device with the operator. According to some exemplary implementations of the present disclosure, after associating the medical device with the operator, the mobile terminal will associate the operations performed by the operator through the medical device thereafter with the operator.

At optional block 1608, if the terminal device determines to stop using the medical device, it will disassociate the medical device from the operator. According to some exemplary implementations of the present disclosure, cessation of use of the medical device may be determined according to at least one of the following: a request for cessation of use of the medical device is received at the terminal device; the length of time that the terminal device being associated with the medical device exceeds a threshold length; the medical device enters a sleep state; and the medical device enters a shutdown state. According to some exemplary implementations of the present disclosure, the mobile terminal may also remind the user who logs in the client system, of the cessation of use of the medical device, for example, through the client system.

Continuing to refer to FIG. 13, when the operator selects the function part 1320 by, for example, touching, the operator enters into client system's supplementary inspection record function. The supplementary inspection record means that when there are operations performed by the medical device that are not associated with the operator, the user can choose to associate these operations with various operators such as himself.

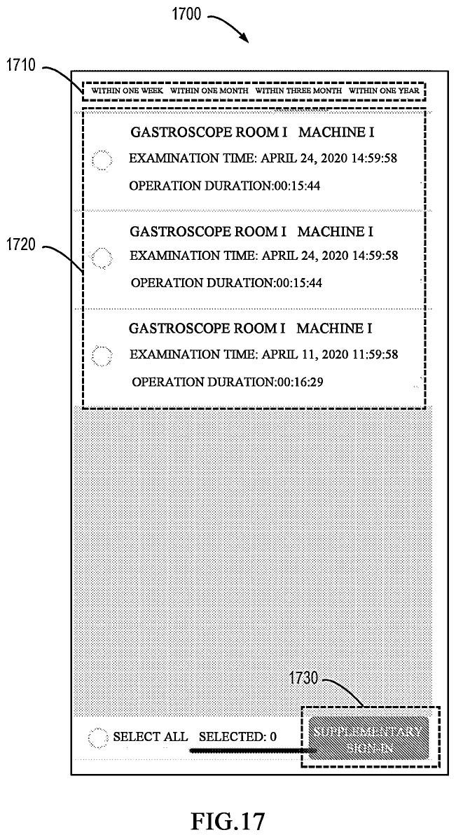

FIG. 17 shows a schematic diagram 1700 when the client system is running. The schematic diagram 1700 is a schematic diagram of the client system associated with the supplementary inspection record function. The schematic diagram 1700 shows three functional parts 1710, 1720 and 1730, which may respectively correspond to different functional modules of the client system.

The function part 1710 may support, for example, in the form of touch to select to display operations performed by the medical device that are not associated with the operator for a period of time. Selectable options may comprise, for example, within one week, within one month, within three months, or within one year.

The function part 1720 can support display of operations performed by the medical equipment associated with the operator. The displayed content can comprise, for example, an examination room where the gastroscope is performed, the number of the gastroscope examination, the time of the examination, and the duration of the examination. The user can select a corresponding operation by, for example, touching.

The functionality part 1720 may support associating selected actions with the user logged into the client system. When the user, for example, touches to select the supplementary sign-in function comprised in the function part 1720, the logged-in client system associates the selected operation with the user.

The operations associated with the diagram 1700 may comprise the user interacting with the functional parts 1710 and 1720 by means of inputting or touching with the client system, thereby entering a specific functional module or calling a specific functional module to display or output information.

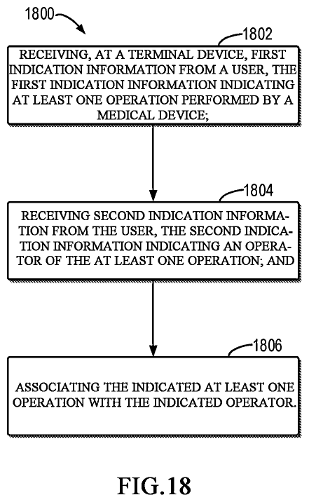

FIG. 18 schematically shows a flowchart of an information processing method 1800 according to an exemplary implementation of the present disclosure. The method 1800 may embody corresponding function of the client system shown in FIG. 17, but may alternatively or additionally comprise other function. The method 1800 may also comprise additional steps not shown and/or steps shown may be omitted, as the scope of the present disclosure is not limited in this respect.

At block 1802, receiving first indication information indicates at least one operation performed by at least one operator through the medical device at the terminal device. This action may correspond to the user selecting a corresponding operation using the function part 1720 by, for example, touching. According to some exemplary implementation manners of the present disclosure, the user may also provide the first indication information to the terminal device through various forms such as voice input.

At block 1804, second indication information is received at the terminal device, where the second indication information indicates the operator. According to some exemplary implementations of the present disclosure, receiving the second indication information at the terminal device may refer to the user logging in to the client system, and the terminal device regards information related to the action of logging-in as the second indication information and regards the user logging-in the client system as the indicated operator. According to some other exemplary implementations of the present disclosure, when the user who logs into the client system is not the operator who performs at least one operation performed by a medical device, but the specific operator can be determined, the user who logs into the client system may send the second indication information to the terminal device to indicate a specific operator.

At block 1806, the indicated at least one operation is associated with the indicated operator. This action is the same as that described above with respect to FIG. 17, and will not be repeated here.

Continuing to refer to FIG. 13, when the operator selects the function part 1330 by, for example, touching, he enters into department examination record function of the client system. The department examination record is used for checking operations performed by a plurality of operators through medical equipment associated with the entire department.

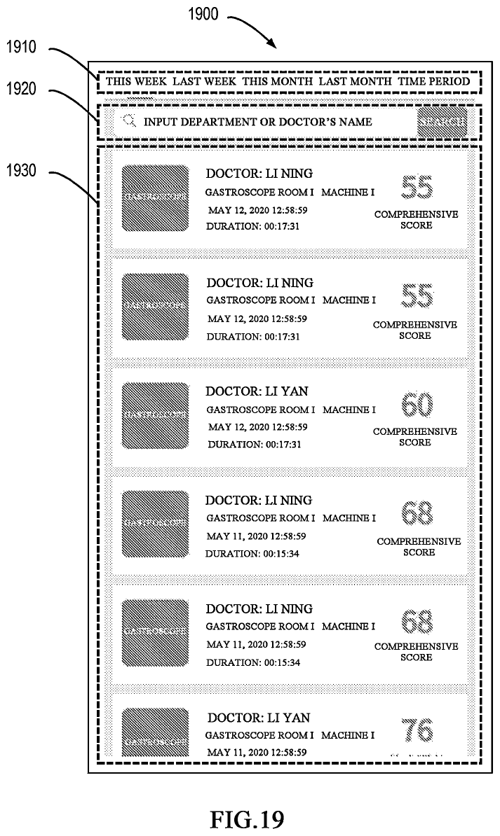

FIG. 19 shows a schematic diagram 1900 when a client system is running. The schematic diagram 1900 is a schematic diagram of department examination record function of the client system'. The schematic diagram 1900 shows three functional parts 1910, 1920 and 1930, which may respectively correspond to different functional modules of the client system.

The function part 1910 may support, for example, in the form of touch to select and display operations performed by multiple operators through the medical device for a period of time. The optional options may comprise, for example, this week, last week, this month, last month, etc., and may support the user of the client system to input a specific time period by means of input.

The function part 1920 can support retrieval of associated operations performed by multiple operators through medical equipment by information such as specific departments or doctors' names, titles, etc., and then only the retrieved operations can be displayed, so that it is possible to view specific operations more efficiently. According to some exemplary implementations of the present disclosure, the client system supports different levels of retrieval for different logged-in users. For example, for hospital-level users, such as hospital administrators, retrieval conditions may comprise departments, titles, and names, so that hospital administrators can query operation records of doctors and nurses in the entire hospital.

For department-level users, for example, department managers, filter conditions do not comprise departments, but only job titles and names, so that department managers can query operation records of doctors and nurses in their departments.

The function part 1930 can support display of operations performed by the medical equipment associated with the operator. The displayed content can comprise, for example, the doctor' name, the examination room where the gastroscope is performed, the gastroscope number, the time of the examination, and operation duration of the examination and scores for this operation, etc. The user can select a corresponding operation by, for example, touching.

Operations associated with the schematic diagram 1900 may comprise the user interacting with the functional parts 1910, 1920, and 1930 by means of inputting or touching through the client system, thereby entering a specific functional module or calling a specific functional module to display or output information. The output information comprises a time period for screening records, a window for searching departments or doctor names, the name of the operator, the medical device number, the date of operation, the duration of the operation, and the overall score. The output information may also be other relevant content used to help check records.

With continued reference to FIG. 13, when the operator selects the function part 1340 by, for example, touching, the operator enters function of My examination list of the client system. My examination list is used to check actions performed by the medical device associated with the user logged into the client system.

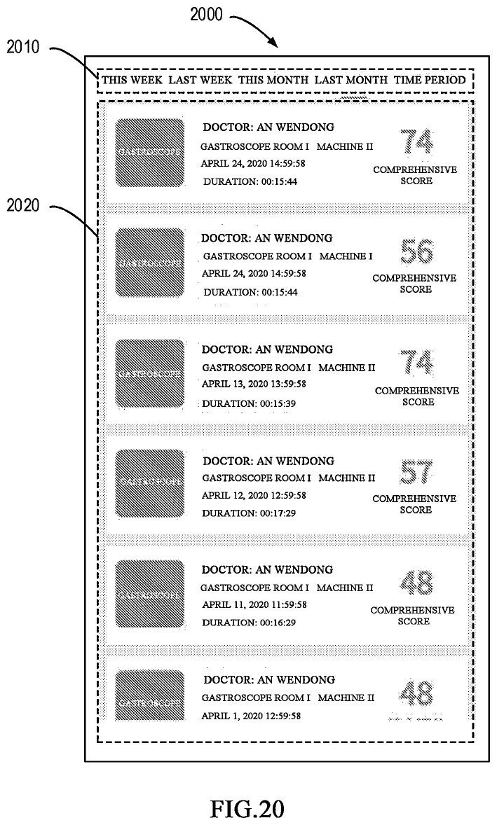

FIG. 20 shows a schematic diagram 2000 when a client system is running. The diagram 2000 is a diagram of the client system associated with the function of My examination list. The schematic diagram 2000 shows two functional parts 2010 and 2020, which may respectively correspond to different functional modules of the client system. The two functional parts 2010 and 2020 respectively correspond to the functional parts 1910 and 1930 described with reference to FIG. 19, and details are not repeated here. It should be pointed out that since the function of My examination list does not need to display operations performed by others through the medical equipment, there is no need for a retrieval function similar to the function 1920 shown in FIG. 19.

Operations associated with the schematic diagram 2000 may comprise the user interacting with the functional parts 2010 and 2020 by means of inputting or touching through the client system, thereby entering a specific functional module or calling a specific functional module to display or output information. The output information comprises the time period of the screening records, the recorder's name, the medical equipment number, the operation date, the operation duration and the comprehensive score. The output information may also be other relevant content used to help check records.

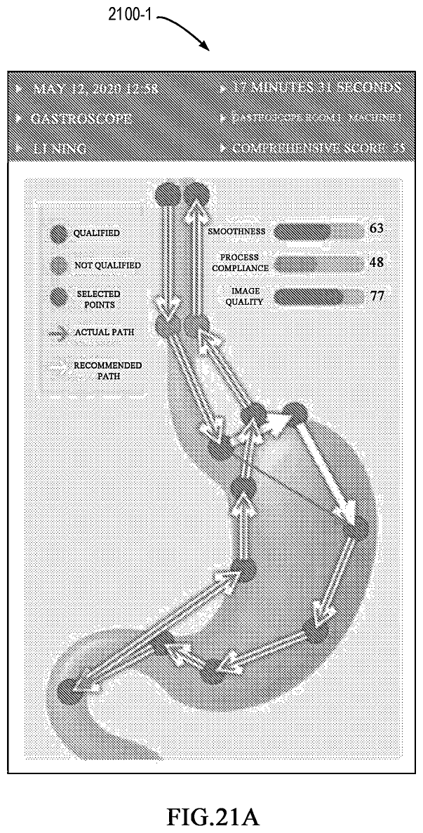

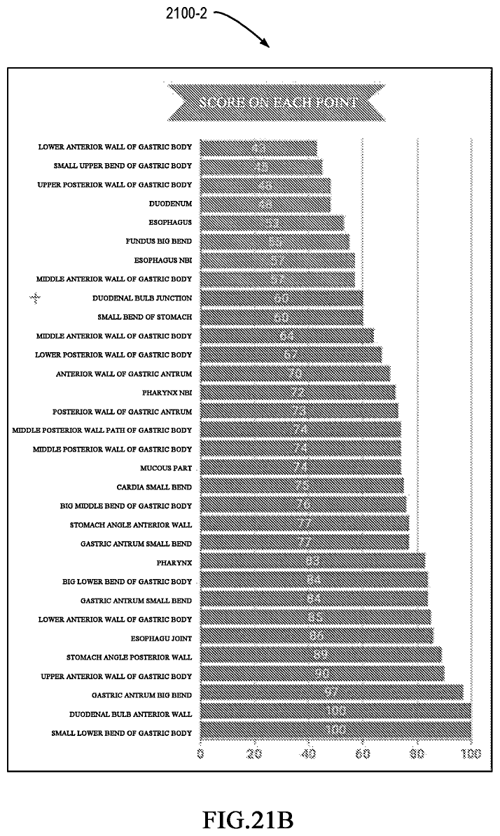

When the user of the client system selects, for example, operations in the schematic diagram 1900 shown in FIG. 19 and the schematic diagram 2000 shown in FIG. 20 by touching, the client system can display specific content about the selected operations, as shown in FIG. 21A and FIG. 21B.

FIGS. 21A-21B show schematic diagrams 2100-1-2100-2 when a client system is running. According to some exemplary implementations of the present disclosure, the schematic diagrams 2100-1 to 2100-2 are respectively parts of the display content about the specific content of selected operations, and they are combined to form complete display content which may be longer and in the form of the image, so that the user can browse the entire display content by scrolling the image. According to some exemplary implementations of the present disclosure, the above two parts may also be displayed in separate interfaces, and the user may switch between the two interfaces, for example, by clicking.

The schematic diagram 2100-1 shows an upper half of the complete display content which comprises the doctor or patient' name, the type of endoscope (in this implementation, gastroscope), the gastroscope room where the gastroscope is performed, the gastroscope number, the date and time of inspection, the duration of the inspection and the score of this operation, etc. In addition, the upper half of the complete display content shown in the schematic diagram 2100-1 also comprises motion track of the gastroscope displayed on the simulated image of the stomach, the actual route and the recommended route, the qualified or unqualified operations and missing points, and scoring for smoothness, process regulation, and image quality.

The diagram 2100-2 shows the lower half of the complete display content which comprises scores for each point examined by the gastroscopy operation.

Operations associated with the schematic diagram 2100 may comprise that the user interacting with the content shown in the schematic diagram 2100 by means of input or touch through the client system to so as to enter a specific functional module or call a specific functional module to display or output information. The output information may comprise basic information, such as the name of the operator, the medical device number, the date of operation, the duration of the operation, and the comprehensive score. Further, the output information comprises an inspection route map, such as a base map of the inspection route, each points, recommended route, actual route, score progress bar and specific scores/scores of each point.

Continuing to refer to FIG. 13, when the operator selects the function part 1350 by, for example, touching, the user enters the My quality control analysis function of the client system. The My examination list function is used to check quality analysis of actions performed by users logged into the client system through medical devices.

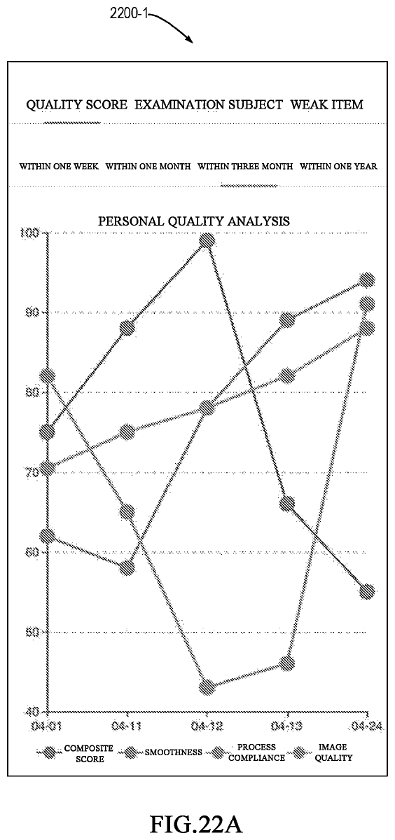

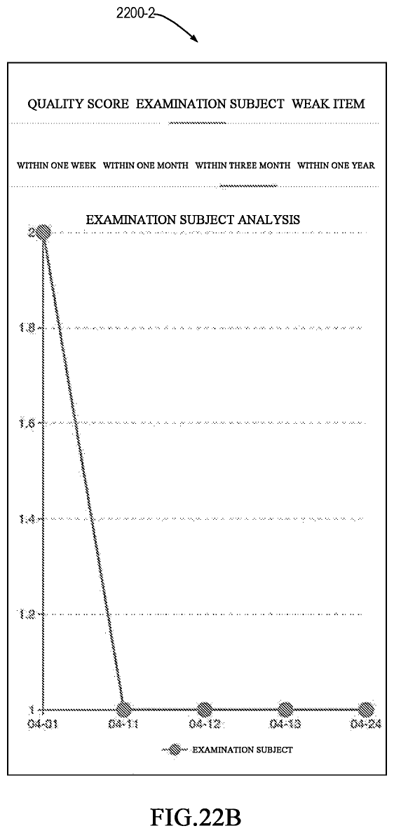

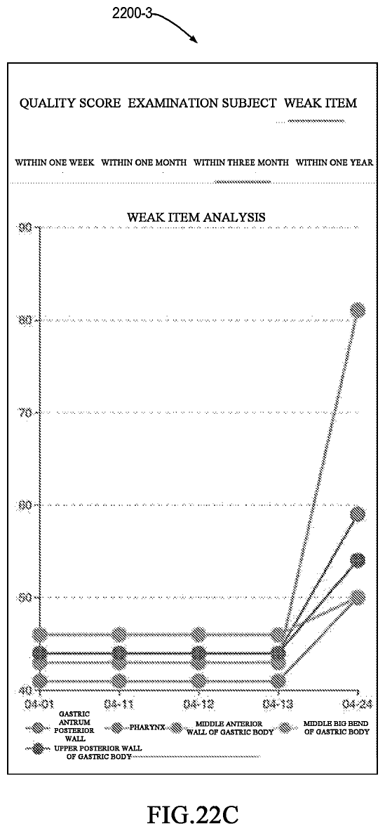

FIGS. 22A-22B show schematic diagrams 2200-1-2200-3 when a client system is running. According to some exemplary implementations of the present disclosure, the schematic diagrams 2100-1 to 2100-3 are respectively parts of the display content about the specific content of selected operations, and they are combined to form complete display content which may be longer and in the form of the image, so that the user can browse the entire display content by scrolling the image. According to some exemplary implementations of the present disclosure, the above three parts may also be displayed in separate interfaces, and the user may switch between the two interfaces, for example, by clicking.

The schematic diagram 2200-1 shows part of the complete display content which comprises personal quality analysis involving quality scoring, in which data within a week, within a month, within three months or within a year can be selected, and different curves can be used to display composite score, smoothness score, process compliance score, and image quality score for an examination performed by the operator using the medical device on coordinates with the date as the X axis and the score as the Y axis.

The diagram 2200-2 shows part of the complete display content which comprises analysis of the number of examination subjects, in which data within one week, one month, to three months or one year can be selected, and a curve can be used to display the number of examinations performed by the operator using the medical device on coordinates with the date as the X axis and the number of examination subjects as the Y axis.

The diagram 2200-3 shows part of the complete display content which comprises weak item analysis, in which data within a week, within a month, within three months or within a year can be selected, and different curves can be used to display scores of the weaker items of the examination performed by the operator using the medical device on coordinates with the date as the X axis and the score as the Y axis. According to some exemplary implementations of the present disclosure, a score threshold may be preset, so that, for example, an item whose average score is lower than the score threshold is determined as a weak item for the operator using the medical device. According to some exemplary implementations of the present disclosure, operators may be ranked with respect to average scores of different items, and items ranked after a threshold value may be selected as weak items of the operators.

Operations associated with the schematic diagram 2200 may comprise that the user interacting with the content shown in the schematic diagram 2100 by means of input or touch through the client system to so as to enter a specific functional module or call a specific functional module to display or output information. The output information in the quality score comprises comprehensive comparison results of comprehensive score, process compliance score, image quality score, smoothness score, and operation time. Average score of the departments or hospitals will also be output for reference; the output information in the number of examination subjects comprises the screened time period, the number of subjects, and the cumulative number of subjects; the output information in weak items comprises the name of the inspection point/site, the corresponding score, and the change trend/curve of the score according to the time relationship.

Continuing to refer to FIG. 13, when the operator selects the function part 1360 by, for example, touching, the operator enters the departmental quality control analysis function of the client system. The department Inspection &Record function is used to inspect quality analysis of operations performed by multiple operators through medical equipment associated with the entire department, which can comprise quality scoring, index comparison, number of subjects and weak items, and statistics belong to a specific time period and desired to be displayed can be selected according to time.

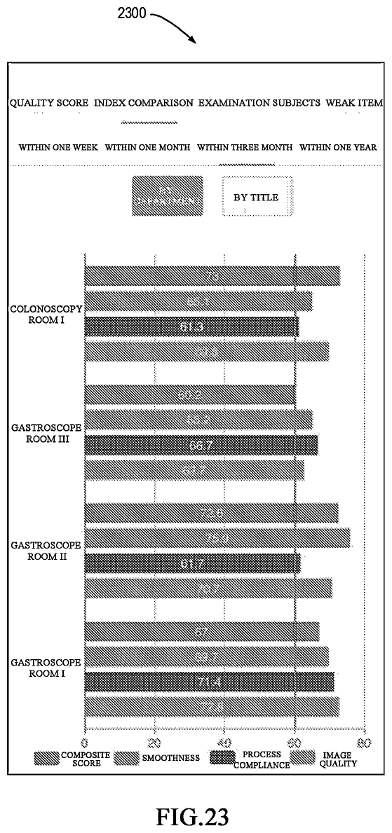

FIG. 23 shows a schematic diagram 2300 when a client system is running. The schematic diagram 2300 shows an index comparison, specifically the sum score, score on a score standard, process compliance score and image quality score of different endoscopy departments classified by department. According to some exemplary implementations of the present disclosure, the scores can also be classified according to job titles.

Operations associated with the schematic diagram 2300 may comprise that the user interacting with the content shown in the schematic diagram 2300 by means of input or touch through the client system to so as to enter a specific functional module or call a specific functional module to display or output information. The output information in the departmental quality control analysis function comprises comprehensive scores, process compliance scores, image quality scores, and smoothness scores according to time periods such as within one week, within one month, within three months, within one year, and according to comprehensive comparison results of departments or titles.

According to some other exemplary implementations of the present disclosure, when the operator selects the function part 1340 to enter the My examination list function of the client system by, for example, touching or selects the function part 1350 to enter the My quality control analysis function of client system by, for example, touching, the corresponding content may not be displayed directly, but optional options may be selected.

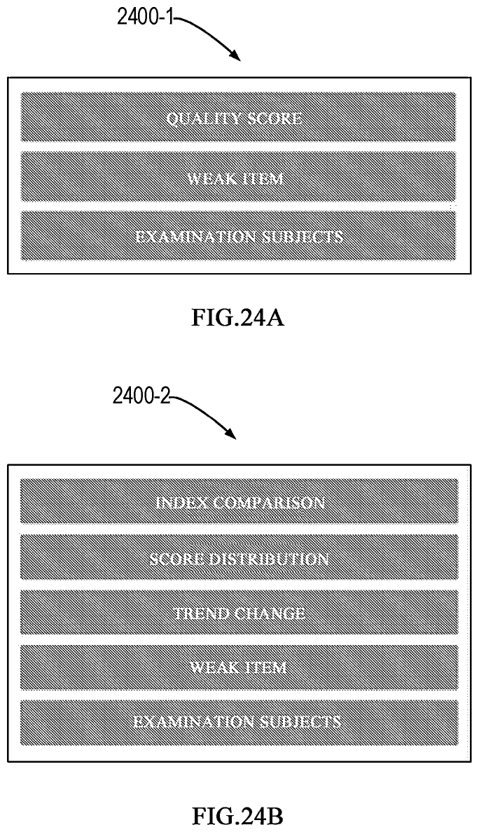

FIGS. 24A to 24B show schematic diagrams 2400-1 to 2400-2 when a client system is running. The schematic diagram 2400-1 shows optional options displayed when the operator selects the function part 1340 to enter the My examination list function of the client system by, for example, touching, comprising quality scores, weak items and the number of examination subjects, and the schematic diagram 2400-2 shows optional options displayed when the operator selects the function part 1350 to enter the My quality control analysis function of the client system by, for example, touching, comprising index comparison, score distribution, trend change, and weak items and the number of examination subjects. Users can enter further display interfaces by selecting these options.

Operations associated with the schematic diagram 2400 may comprise that the user interacting with the content shown in the schematic diagram 2400 by means of input or touch through the client system to so as to enter a specific functional module or call a specific functional module to display or output information.

According to some exemplary implementations of the present disclosure, in the scenario involved in FIG. 24B, the client system may provide different levels of information for different logged-in users. For example, for hospital-level users, such as hospital administrators, retrieval conditions may comprise departments, titles, and names, so that hospital administrators can obtain quality control analysis on operation records of doctors and nurses in the entire hospital. For department-level users, for example, department managers, only quality control analysis on operation records of doctors and nurses in their departments can be provided.

Continuing to refer to FIG. 24A, when the operator selects the weak item option by, for example, touching, the user enters the quality scoring function in the My quality control analysis function of the client system.

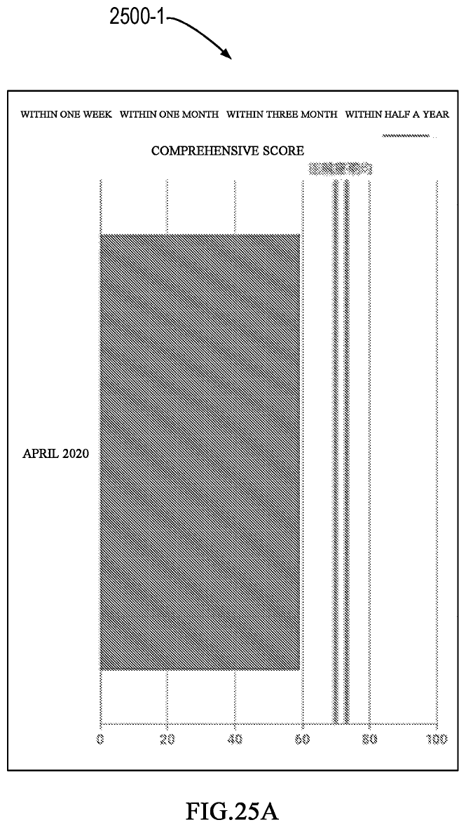

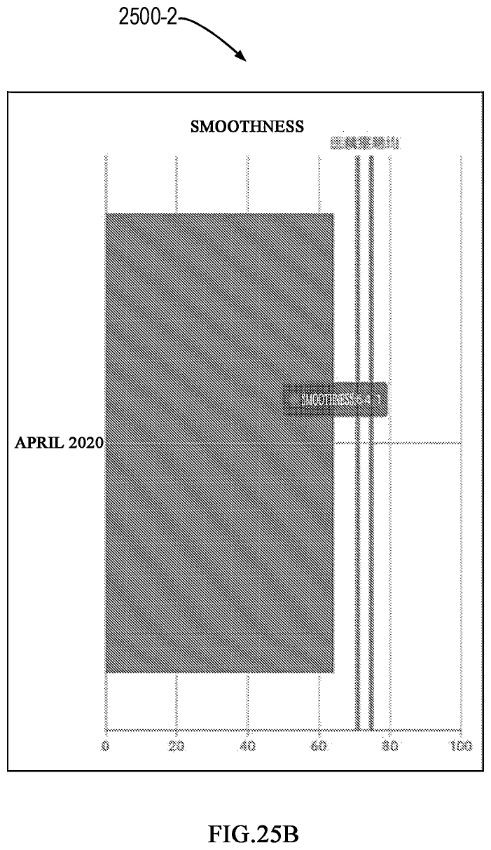

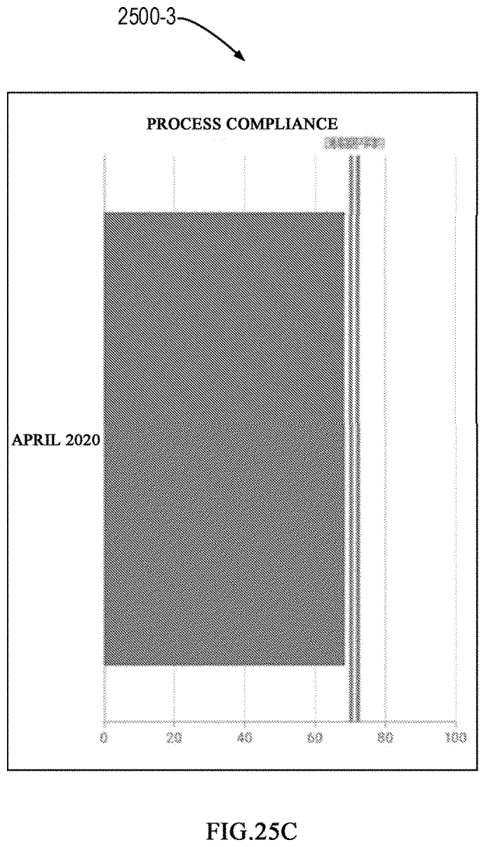

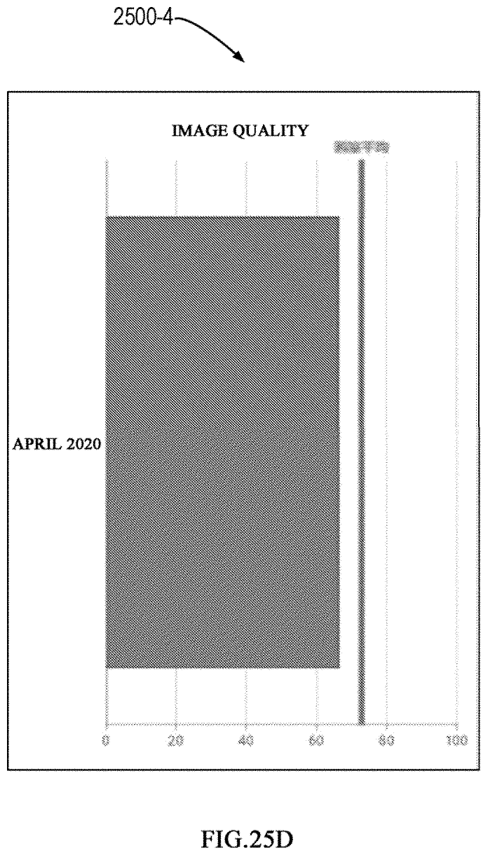

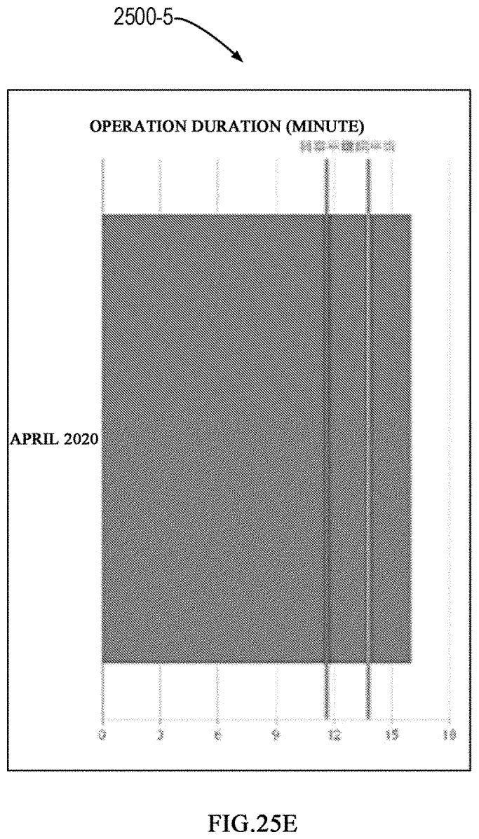

FIGS. 25A-25E show schematic diagrams 2500-1-2400-5 when a client system is running, corresponding to the quality scoring function in the My quality control analysis function of the client system. The schematic diagrams 2500-1 to 2500-5 respectively show composite score, smoothness score, process compliance score, and image quality score of the operators of the medical device and the operation time on coordinates with the X-axis as the score and the Y-axis as the corresponding time period, and can show average score of the department or hospital to which the operators belong. According to some exemplary implementations of the present disclosure, the diagrams 2500-1 to 2500-5 are respectively part of the display content about the quality score, and they are combined to form complete display content which may be longer and in the form of the image, so that the user can browse the entire display content by scrolling the image. According to some exemplary implementations of the present disclosure, the above five parts may also be displayed in separate interfaces, and the user may switch between the two interfaces, for example, by clicking.

Operations associated with the schematic diagram 2500 may comprise that the user interacting with the content shown in the schematic diagram 2500 by means of input or touch through the client system to so as to enter a specific functional module or call a specific functional module to display or output information. The output information of the My quality control analysis may comprise composite score, process compliance score, and image quality score, smoothness score and operation time score according to the comprehensive comparison result of the time period within one week, within one month, within three months, within one year and the average score of the department or hospital.

Continuing to refer to FIG. 24A, when selecting the weak items by, for example, touching, the operator enters the weak items of the My quality control analysis function of the client system.

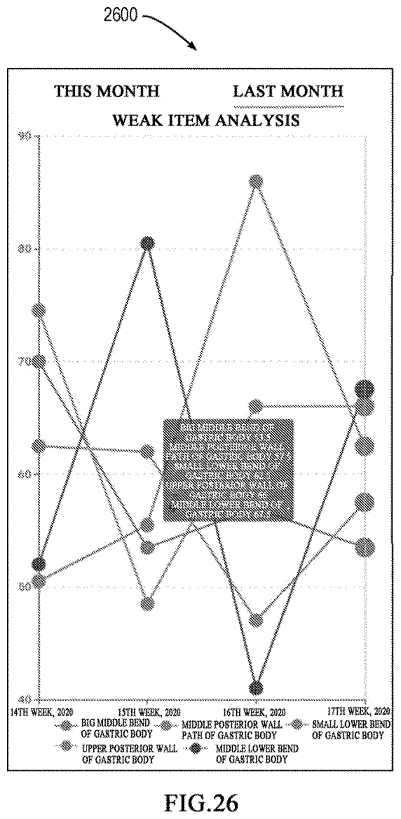

FIG. 26 shows a schematic diagram 2600 when a client system is running, corresponding to the weak item function in the My quality control analysis function of the client system. The content of the schematic diagram 2600 is similar to that of the schematic diagram 2200-3 described above with respect to FIG. 22C, and will not be repeated here.

Operations associated with the schematic diagram 2600 may comprise that the user interacting with the content shown in the schematic diagram 2600 by means of input or touch through the client system to so as to enter a specific functional module or call a specific functional module to display or output information. The output information in the weak items comprises the name of the examination point/site, and the corresponding score according to the time period such as this month and last month and the change trend/curve in units such as weeks or days

Continuing to refer to FIG. 24A, when selecting the examination subjects by, for example, touching, the operator enters the examination subjects function of the My quality control analysis function of the client system.

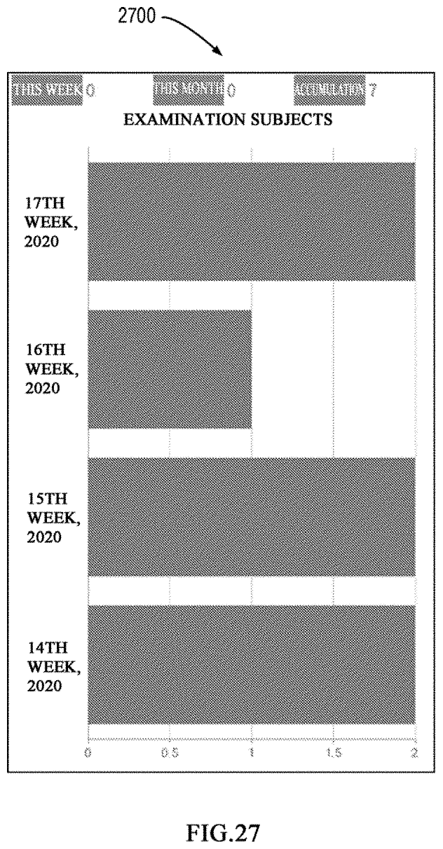

FIG. 27 shows a schematic diagram 2700 when a client system is running, corresponding to examination subjects function of the My quality control analysis function of the client system. The schematic diagram 2700 can display examination subjects in a certain time period or the cumulative number of examination subjects.

Operations associated with the schematic diagram 2700 may comprise that the user interacting with the content shown in the schematic diagram 2700 by means of input or touch through the client system to so as to enter a specific functional module or call a specific functional module to display or output information. The output information in the examination subjects function comprises a change trend/histogram of the number of examination subjects according to, for example, this week, this month, the accumulated time period, and, for example, in units of weeks.

Continuing to refer to FIG. 24B, when selecting the index comparison by, for example, touching, the operator enters the index comparison function of the department quality control analysis function of the client system.

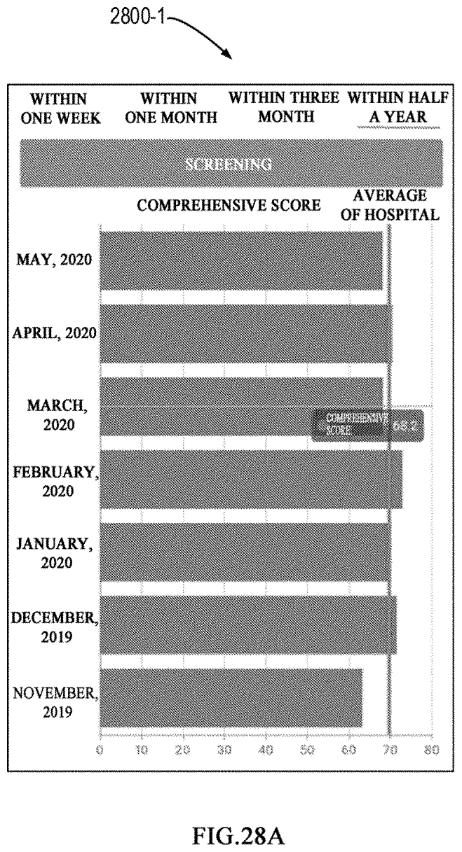

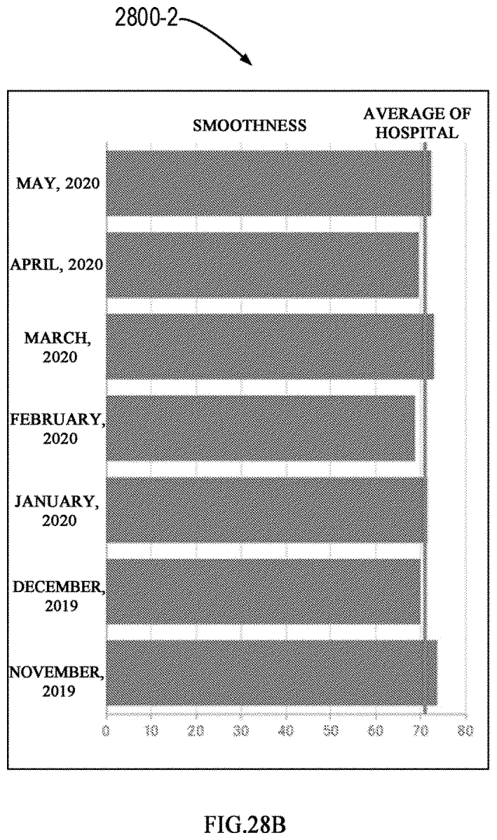

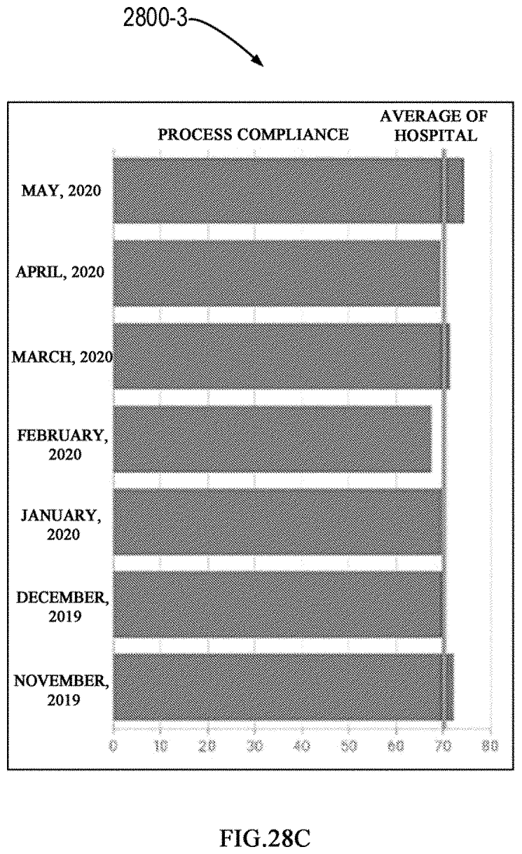

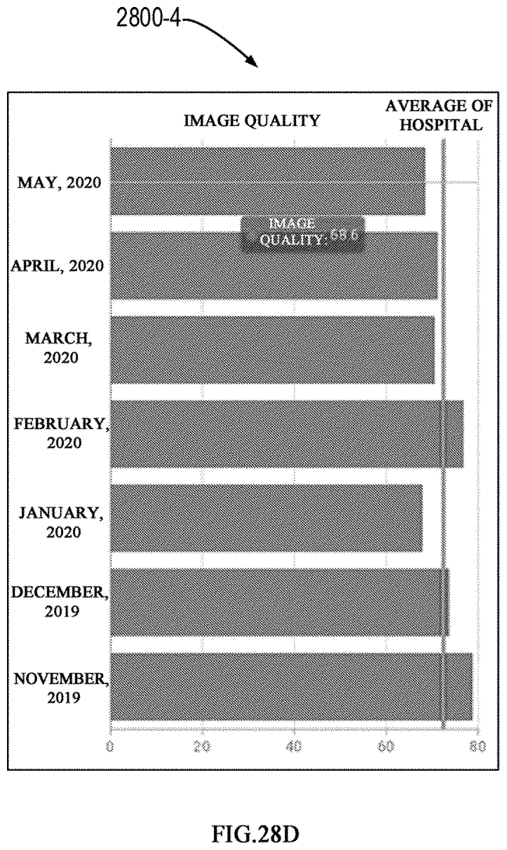

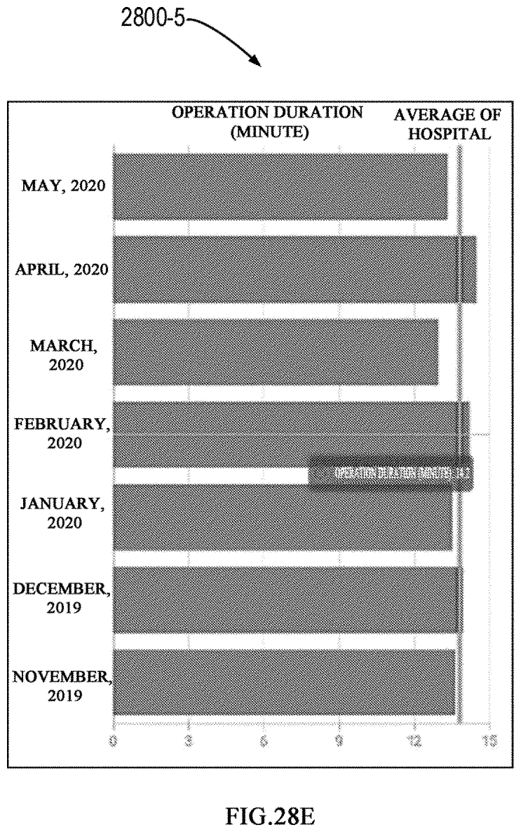

FIGS. 28A-28E show schematic diagrams 2800-1-2800-5 when a client system is running, corresponding to the index comparison function of the department quality control analysis function of the client system. The schematic diagrams 2800-1-2800-5 respectively show composite score, smoothness score, process compliance score, and image quality score of the operators of the medical device and the operation time on coordinates with the X-axis as the score and the Y-axis as the corresponding time period, and can show average score of the hospital to which the operators belong. According to some exemplary implementations of the present disclosure, the diagrams 2800-1-2800-5 are respectively part of the display content about the index comparison, and they are combined to form complete display content which may be longer and in the form of the image, so that the user can browse the entire display content by scrolling the image. According to some exemplary implementations of the present disclosure, the above five parts may also be displayed in separate interfaces, and the user may switch between the two interfaces, for example, by clicking.

Operations associated with the schematic diagram 2800 may comprise that the user interacting with the content shown in the schematic diagram 2800 by means of input or touch through the client system to so as to enter a specific functional module or call a specific functional module to display or output information. The output information of the index comparison may comprise composite score, process compliance score, and image quality score, and smoothness score according to the comprehensive comparison result of the time period within one week, within one month, within three months, within one year, the change trend/histogram in units of weeks, and comparison of average level of the hospital.

Continuing to refer to FIG. 24B, when selecting the score distribution by, for example, touching, the operator enters the score distribution function of the department quality control analysis function of the client system.

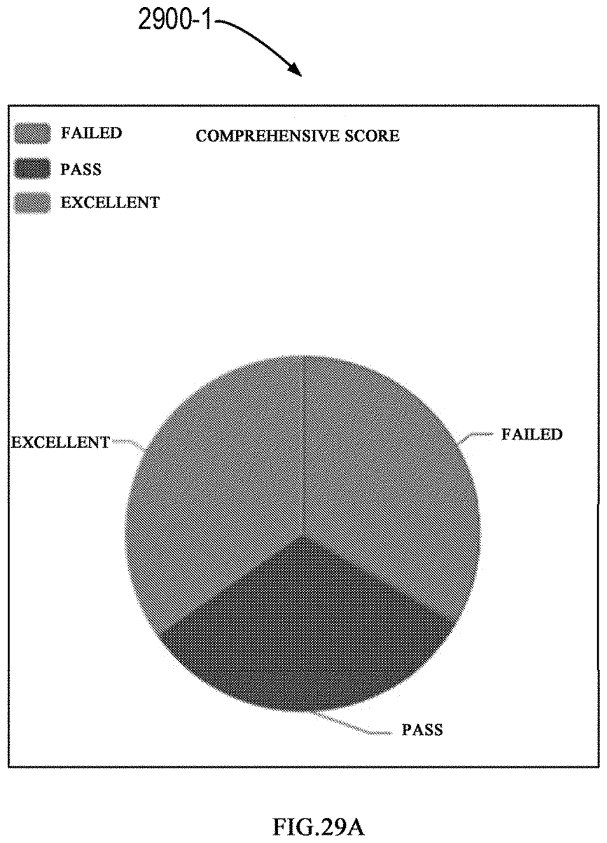

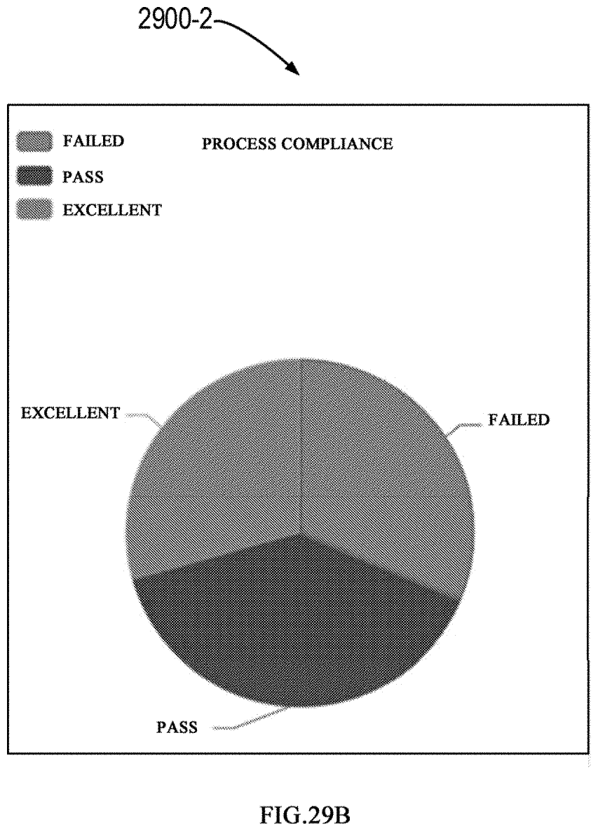

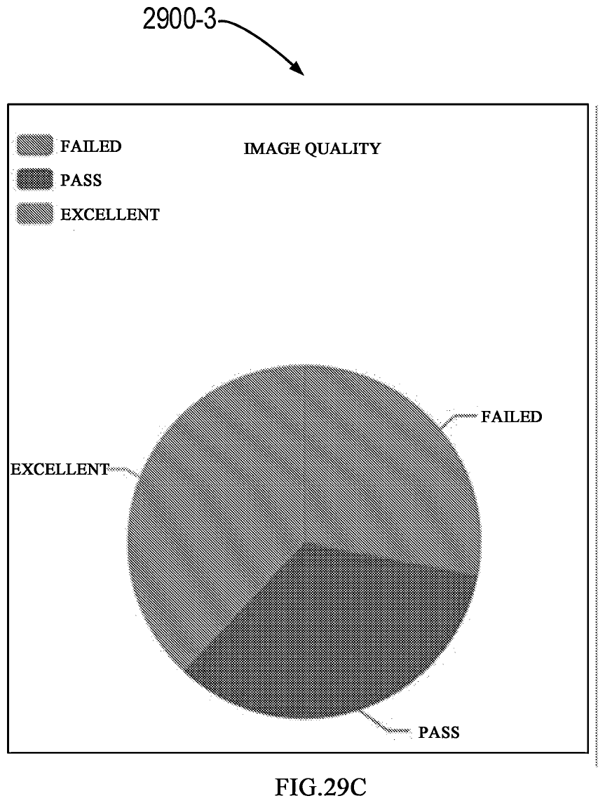

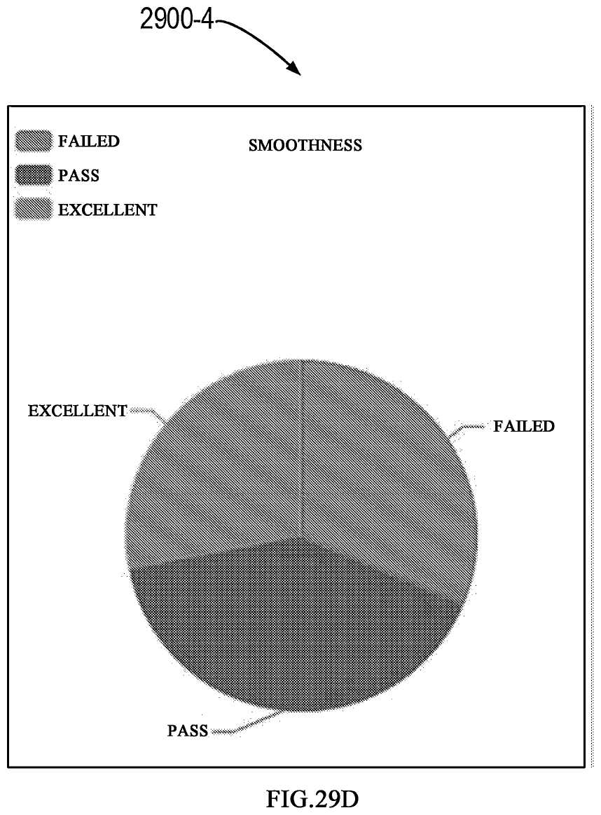

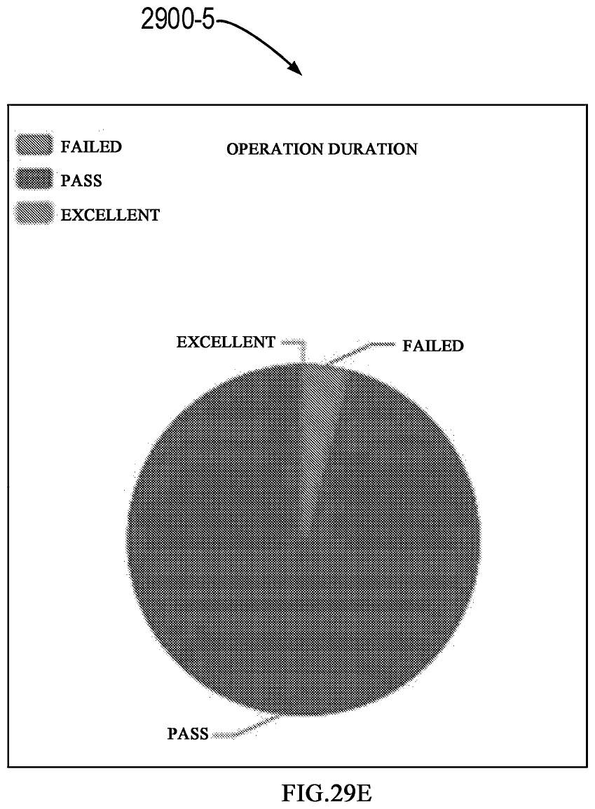

FIGS. 29A-29E show schematic diagrams 2900-1-2900-5 when a client system is running, corresponding to the score distribution function of the department quality control analysis function of the client system. The schematic diagrams 2900-1-2900-5 respectively show composite score, smoothness score, process compliance score, image quality score, and ratio of the excellent, the passed and the failed of the operators of the medical device classified by departments. According to some exemplary implementations of the present disclosure, the diagrams 2900-1-2900-5 are respectively part of the display content about the score distribution, and they are combined to form complete display content which may be longer and in the form of the image, so that the user can browse the entire display content by scrolling the image. According to some exemplary implementations of the present disclosure, the above five parts may also be displayed in separate interfaces, and the user may switch between the two interfaces, for example, by clicking. According to some exemplary implementations of the present disclosure, the user may click on the icon to display further information.

Operations associated with the schematic diagram 2900 may comprise that the user interacting with the content shown in the schematic diagram 2900 by means of input or touch through the client system to so as to enter a specific functional module or call a specific functional module to display or output information. The output information of the score distribution may comprise composite score, process compliance score, and image quality score, and smoothness score,

Continuing to refer to FIG. 24B, when selecting the trend change by, for example, touching, the operator enters the trend change function of the department quality control analysis function of the client system.

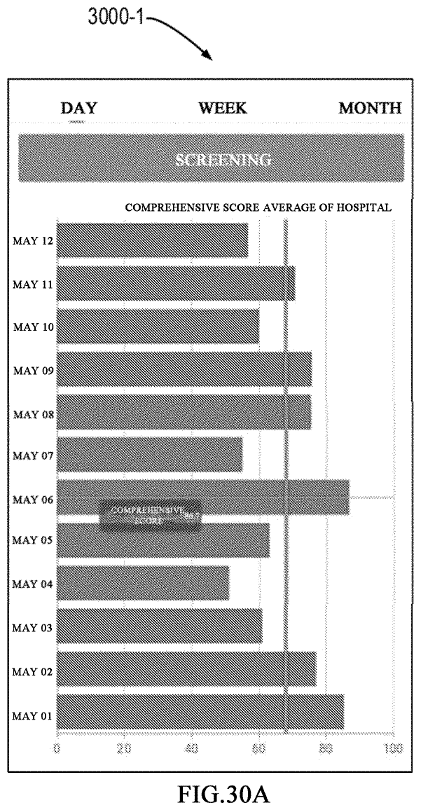

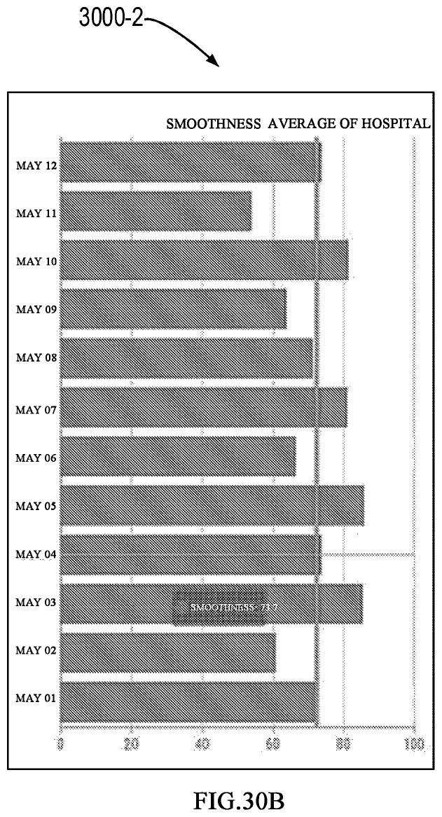

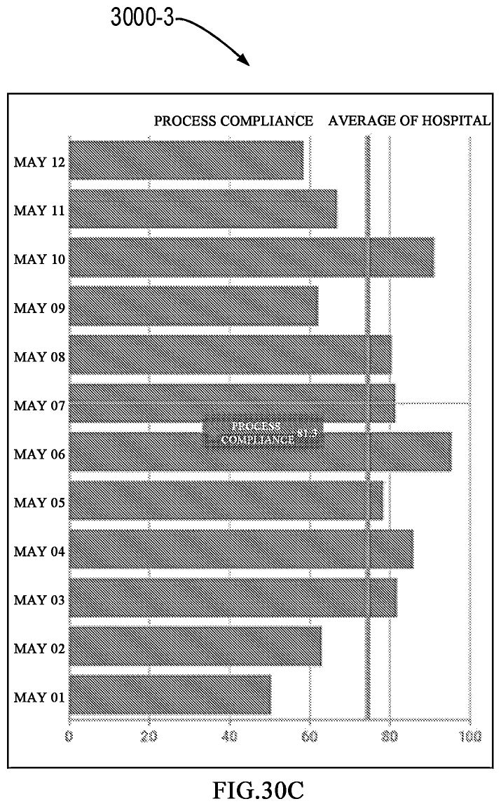

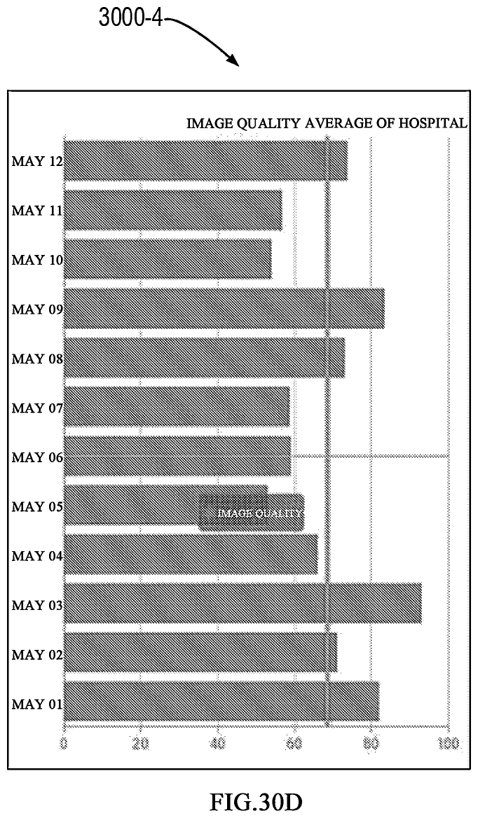

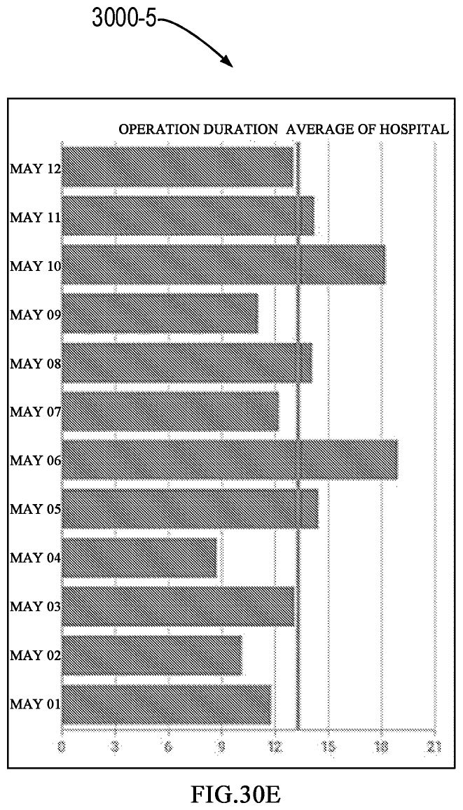

FIGS. 30A-30E show schematic diagrams 3000-1-3000-5 when a client system is running, corresponding to the trend change function of the department quality control analysis function of the client system. The schematic diagrams 3000-1-3000-5 respectively show composite score, smoothness score, process compliance score, image quality score, and operation time of the operators of the medical device classified by departments, and can show the average score of the hospital, so that the change trend of the above scores can be understood. According to some exemplary implementations of the present disclosure, the diagrams 3000-1-3000-5 are respectively part of the display content about the trend change, and they are combined to form complete display content which may be longer and in the form of the image, so that the user can browse the entire display content by scrolling the image. According to some exemplary implementations of the present disclosure, the above five parts may also be displayed in separate interfaces, and the user may switch between the two interfaces, for example, by clicking. According to some exemplary implementations of the present disclosure, the user may click on the icon to display further information.

Operations associated with the schematic diagram 3000 may comprise that the user interacting with the content shown in the schematic diagram 3000 by means of input or touch through the client system to so as to enter a specific functional module or call a specific functional module to display or output information. The output information of the change trend may comprise composite score, process compliance score, and image quality score, and smoothness score according to the time period within one day, one week, and one month, and the change trend/histogram in units of days, and comparison of average level of the hospital.

Continuing to refer to FIG. 24B, when selecting the weak items by, for example, touching, the operator enters the weak item function of the department quality control analysis function of the client system.

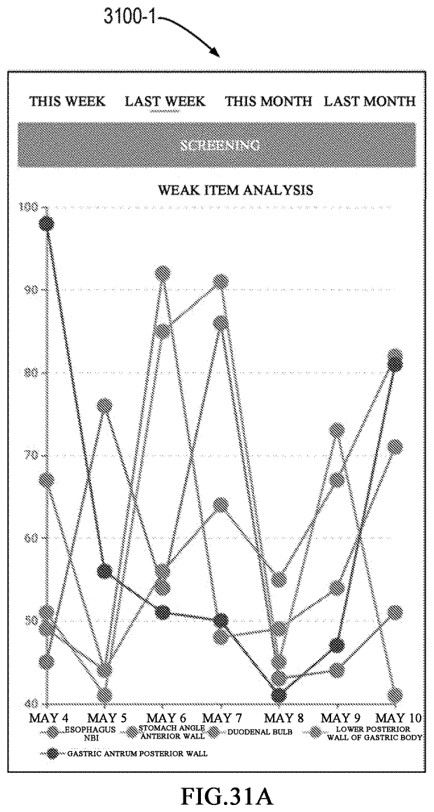

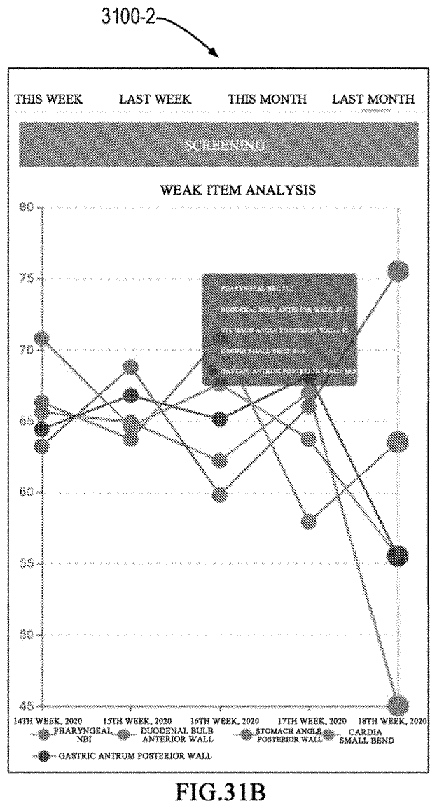

FIGS. 31A-31B show schematic diagrams 3100-1-3100-2 when a client system is running, corresponding to the weak item function of the department quality control analysis function of the client system. Data within this week, the last week (the schematic diagram 3100-1), this month or the last month (the schematic diagram 3100-2) can be selected, and different curves can be used to display scores of the weaker items of the examination performed by the operator using the medical device on coordinates with the date or the week as the X axis and the score as the Y axis. According to some exemplary implementations of the present disclosure, a score threshold may be preset, so that, for example, an item whose average score is lower than the score threshold is determined as a weak item for the operator using the medical device. According to some exemplary implementations of the present disclosure, operators may be ranked with respect to average scores of different items, and items ranked after a threshold value may be selected as weak items of the operators.

Operations associated with the schematic diagram 3100 may comprise that the user interacting with the content shown in the schematic diagram 3100 by means of input or touch through the client system to so as to enter a specific functional module or call a specific functional module to display or output information. The output information in the weak item function comprises name of the inspection point/site, the corresponding score, and the change trend/curve of the score according to the time relationship.

Continuing to refer to FIG. 24B, when selecting the examination subjects by, for example, touching, the operator enters the examination subjects function of the department quality control analysis function of the client system.

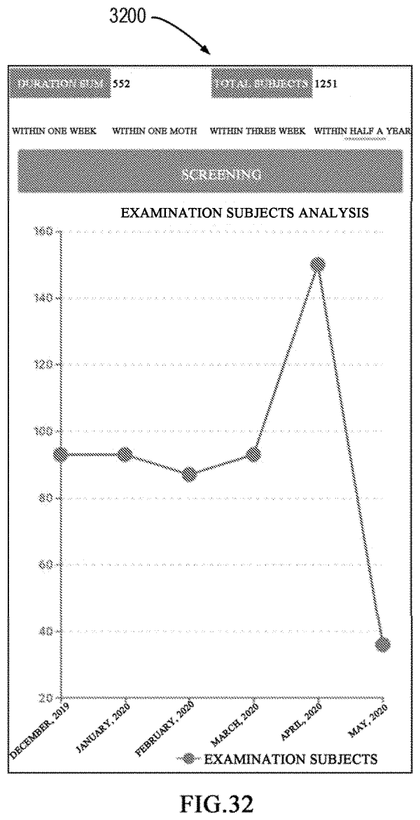

FIG. 32 shows a schematic diagram 3200 when a client system is running, comprising examination subject analysis. Data within one week, one month, three months or half a year can be selected, and different curves can be used to display the number of the examination performed by the operator using the medical device classified by departments on coordinates with the date or the week as the X axis and the examination subject as the Y axis.

Operations associated with the schematic diagram 3200 may comprise that the user interacting with the content shown in the schematic diagram 3200 by means of input or touch through the client system to so as to enter a specific functional module or call a specific functional module to display or output information. The output information of the examination subject may comprise comparison of the screened time period, the number of the examined subjects and the time.

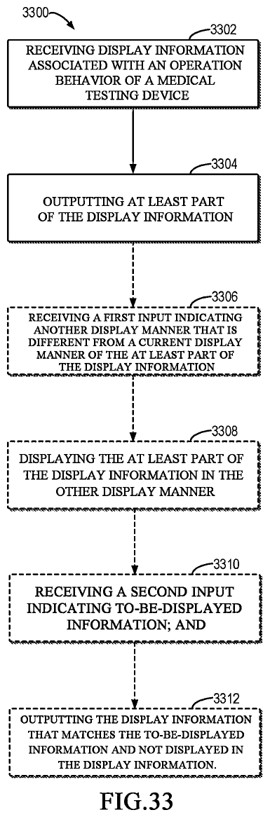

FIG. 33 schematically shows a flowchart of an information processing method 3300 according to an exemplary implementation of the present disclosure. The method 3300 may embody corresponding function of the client system shown in FIG. 13 and FIG. 19 to FIG. 32, but may alternatively or additionally comprise other function. The method 3300 may also comprise additional steps not shown and/or steps shown may be omitted, as the scope of the present disclosure is not limited in this regard.

At block 3302, the terminal device receives first information associated with an operation behavior of a medical testing device. According to some exemplary implementations of the present disclosure, the first information being associated with data collection performed by the medical testing device during operation.

According to some exemplary implementations of the present disclosure, before receiving the first information, the terminal device first receives an instruction from an user using the mobile device, and may send a request for the first information to the cloud storage device, and then receives the first information from the cloud storage device. According to some exemplary implementations of the present disclosure, the first information may be transmitted from the data analysis device of the medical testing device to the cloud storage device, and the first information is determined based on input data from the endoscope of the medical testing device.

According to some exemplary implementations of the present disclosure, the first information received by the terminal device associated with the operation behavior of the medical testing device may comprise at least one of the following: a set of locations of the medical testing device during the operation; sequence information associated with the set of locations; time information associated with the set of locations; trajectory of the medical testing device during the operation; recommended trajectory of the medical testing device during the operation; a deviation degree of the trajectory from the recommended trajectory; speed information of the medical testing device during the operation; smoothness of the medical testing device during the operation; image data collected by the medical testing device during the data collection; image quality of the image data; an organ image of an organ for which the operation is directed; a degree of qualification of a to-be-tested part of the medical testing device to medical testing device examination; a score of the operation behavior; statistical information of the operation behavior; comparative information of the statistical information; suggestions of improvement to the operation behavior; comparison of the different first information for the different operation behavior; and determining the operation behavior according to the score and a score threshold.

In the first information above, according to some exemplary implementations of the present disclosure, the image quality can be determined by at least one of the following: clarity of the image data, the amount of image data, and coverage of the to-be-tested part of the medical testing device covered by the image data.

In the first information above, according to some exemplary implementations of the present disclosure, the statistical information comprises at least one of the following: a quantity of image data collected by the operation behavior in at least a portion of the set of locations; division of the operation behavior according to different operators of the medical testing device; and division of the operation behavior according to organizations to which different operators of the medical testing device belong.

The first information comprises information directly recorded by the endoscope, such as a set of locations of the medical detection equipment during the operation, sequence information associated with the set of locations, time information associated with the set of locations, instantaneous speed of the endoscope during the operation, image data collected by the medical detection equipment during data collection, and information directly recorded by the above-mentioned endoscope received by the processing device 1122, and first processing device information obtained from processing analysis, such as the trajectory the medical testing device during the operation, the recommended trajectory of the medical testing device during the operation, the deviation degree of the trajectory from the recommended trajectory; the speed information of the medical testing device during the operation; the smoothness of the medical testing device during the operation; the image quality of the image data; the degree of qualification of a to-be-tested part of the medical testing device to medical testing device examination; further comprises the statistical information obtained by the processing device 1122 through the statistical analysis of the information of the first processing device, or referred to as the second processing device information, and the second processing device information comprises the a score of the operation behavior; the statistical information of the operation behavior; the comparative information of the statistical information; the suggestions of improvement to the operation behavior; the comparison of the different first information for the different operation behavior; and determining the operation behavior according to the score and a score threshold.

Further, the statistical information in the second processing device information may further comprise statistical information comprising at least one of the following: a quantity of image data collected by the operation behavior in at least part of the set of locations; division of the operation behavior according to different operators of the medical testing device; and division of the operation behavior according to organizations to which different operators of the medical testing device belong.

According to some exemplary implementations of the present disclosure, the endoscope 1110 collects relevant information during the doctor's examination, and then obtains analysis and comparison data according to the doctor's behavior standards stipulated by the hospital. The analysis and comparison can be calculated for whether the doctor's operation behavior conforms to the operating norms/standards, and due to multiple similar calculation, long-term operational records of a certain doctor and operational records of departments and hospitals can be obtained, and statistical processing can be performed on the basis of individuals, departments, and hospitals, so as to provide data support for evaluation and consideration of individuals, departments, and hospitals.

At block 3304, the terminal device outputs the at least part of the first information.

According to some exemplary implementations of the present disclosure, outputting the at least part of the first information comprises at least one of the following: displaying the at least part of the first information; and printing the at least part of the first information.

According to some exemplary implementations of the present disclosure, outputting at least part of the first information comprises: displaying the at least part of the first information in at least one of the following display manners: multi-view switching display, video display, virtual reality display, augmented reality display, and 3D display.

According to some exemplary implementations of the present disclosure, outputting the at least part of the first information comprises: selecting a display manner based on a type of the first information; and displaying the at least part of the first information in the selected display manner. For example, when the type of the first information is the moving trajectory of the endoscope, the 3D display may be selected as the display manner to display the trajectory, so that the trajectory can be viewed more intuitively.

According to some exemplary implementations of the present disclosure, outputting the at least part of the first information comprises at least one of the following: outputting at least part of the set of locations in association with the organ image; and outputting at least part of the trajectory in association with the organ image.

According to some exemplary implementations of the present disclosure, outputting the at least part of the first information comprises at least one of the following: outputting the trajectory and the recommended trajectory in association; and outputting the trajectory and the deviation degree in association. This makes it possible to visually see deficiencies in the performed endoscopic operations.

According to some exemplary implementations of the present disclosure, outputting the at least part of the first information comprises: receiving an input instruction for the first information; and determining the part of the first information in response to the input instruction. After screening operation, the user can see the required information more intuitively, so that the effect of quality control can be better achieved. According to some exemplary implementations of the present disclosure, the input instruction further comprises a screening instruction, and the screening instruction comprises screening conditions comprising at least one of the following: an operator identification of an operator associated with an operation behavior of the medical testing device, an organization identification of an organization to which an operator of the medical testing device belongs, a date the operation behavior occurred, a time period during which the operation behavior occurred, a score threshold of scoring to the operation behavior, a degree of qualification threshold of a degree of qualification of a to-be-tested part of the medical testing device to medical testing device examination, a statistical information threshold of statistical information of the operation behavior and an image quality threshold.

With continued reference to FIG. 33, at optional block 3306, the terminal device receives a first input indicating another display manner that is different from a current display manner of the at least part of the first information. According to some exemplary implementations of the present disclosure, the user of the terminal device may select a display manner of the first information, for example, by using a histogram or a pie chart to display the first information according to a personal preference or preferred display manner.

At optional block 3308, the terminal device displays the first information according to another display manner indicated in optional block 3306 and indicated by the first input.

At optional block 3310, the terminal device receives a second input indicating to-be-displayed information. According to some exemplary implementations of the present disclosure, referring to FIGS. 13 to 32, the user of the terminal device may select to display further information, for example, by touching on the first information. For example, the user may click a certain point on a trajectory of the first information with a finger to display score and suggestion associated with the point.

At optional block 3312, the terminal device outputs the first information that matches the to-be-displayed information and not displayed in the first information according to the to-be-displayed information indicated by the second input and indicated in the optional block 3306

It should be understood that the various numbers and values used in the above-mentioned drawings and descriptions of the present disclosure are only examples, and are not intended to limit the protection scope of the present disclosure. The above numbers and values can be set arbitrarily according to needs, without affecting the normal implementation of the embodiments of the present disclosure.

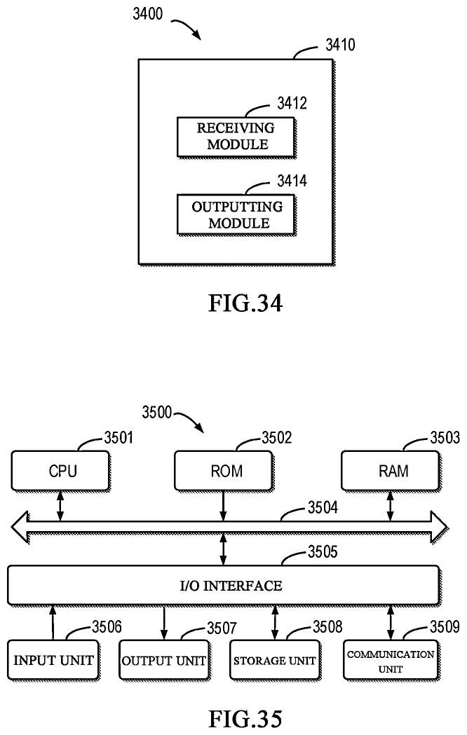

Details of the information processing method have been described above with reference to FIG. 13 and FIGS. 19 to 33. Hereinafter, each block in the information processing device will be described with reference to FIG. 34. FIG. 34 schematically shows a block diagram 3400 of an information processing apparatus 3410 according to an exemplary implementation of the present disclosure. As shown in FIG. 34, an information processing apparatus 3410 is provided, comprising: a receiving module 3412 configured to receive first information associated with the operation behavior of the medical testing device, the first information being associated with data collection performed by the medical testing device during operation; and an output module 3414 configured to output the at least a portion of the first information. According to some exemplary implementations of the present disclosure, the information processing device 3410 is configured to execute specific steps of the above information processing method 3300 shown in FIG. 33.

Through the above description with reference to FIG. 12 to FIG. 33, the technical solution according to the embodiment of the present disclosure has many advantages compared with the traditional solution. For example, using this technical solution, quality control of the operation behavior associated with the inspection can be carried out, so that the quality of the result obtained by the operation behavior, the deviation from the recommended operation, the suggested modification direction and any possible statistical information can be displayed to help doctors improve operations with medical testing device.

FIG. 35 shows a schematic block diagram of an example device 3500 for implementing embodiments of the present disclosure. For example, a computing device 130 shown in FIG. 1 and the data device 1121, the processing device 1122 and the terminal device 1123 shown in FIG. 11 may be implemented by the device 3500. As shown, the device 3500 comprises a central process unit (CPU) 3501, which can execute various suitable actions and processing based on the computer program instructions stored in the read-only memory (ROM) 3502 or computer program instructions loaded in the random-access memory (RAM) 1403 from a storage unit 3508. The RAM 3503 can also store all kinds of programs and data required by the operations of the device 3500. CPU 3501, ROM 3502 and RAM 3503 are connected to each other via a bus 3504. The input/output (I/O) interface 3505 is also connected to the bus 3504.

A plurality of components in the device 3500 is connected to the I/O interface 3505, comprising: an input unit 3506, such as keyboard, mouse and the like; an output unit 3507, e.g., various kinds of display and loudspeakers etc.; a storage unit 1408, such as magnetic disk and optical disk etc.; and a communication unit 3509, such as network card, modem, wireless transceiver and the like. The communication unit 3509 allows the device 3500 to exchange information/data with other devices via the computer network, such as Internet, and/or various telecommunication networks.

The above described each procedure and processing, such as the method 1600, 1800 and 3300, can also be executed by the processing unit 3501. For example, in some embodiments, the method 1600, 1800 and 3300 can be implemented as a computer software program tangibly comprised in the machine-readable medium, e.g., storage unit 3508. In some embodiments, the computer program can be partially or fully loaded and/or mounted to the device 3500 via ROM 3502 and/or communication unit 3509. When the computer program is loaded to RAM 3503 and executed by the CPU 3501, one or more steps of the above described method 1600, 1800 and 3300 can be implemented.

According to some exemplary implementations of the present disclosure, an information processing device is provided, comprising: at least one processing unit; at least one memory coupled to the at least one processing unit and storing instructions for execution by the at least one processing unit that, when executed by the at least one processing unit, cause the device to perform the method 1600 as described above.

According to some exemplary implementations of the present disclosure, an information processing device is provided, comprising: at least one processing unit; at least one memory coupled to the at least one processing unit and storing instructions for execution by the at least one processing unit that, when executed by the at least one processing unit, cause the device to perform the method 1800 as described above.

According to some exemplary implementations of the present disclosure, an information processing device is provided, comprising: at least one processing unit; at least one memory coupled to the at least one processing unit and storing instructions for execution by the at least one processing unit that, when executed by the at least one processing unit, cause the device to perform the method 3300 as described above.

The present disclosure can be method, apparatus, system and/or computer program product. The computer program product can comprise a computer-readable storage medium, on which the computer-readable program instructions for executing various aspects of the present disclosure are loaded.

The computer-readable storage medium can be a tangible apparatus that maintains and stores instructions utilized by the instruction executing apparatuses. The computer-readable storage medium can be, but not limited to, such as electrical storage device, magnetic storage device, optical storage device, electromagnetic storage device, semiconductor storage device or any appropriate combinations of the above. More concrete examples of the computer-readable storage medium (non-exhaustive list) comprise: portable computer disk, hard disk, random-access memory (RAM), read-only memory (ROM), erasable programmable read-only memory (EPROM or flash), static random-access memory (SRAM), portable compact disk read-only memory (CD-ROM), digital versatile disk (DVD), memory stick, floppy disk, mechanical coding devices, punched card stored with instructions thereon, or a projection in a slot, and any appropriate combinations of the above. The computer-readable storage medium utilized here is not interpreted as transient signals per se, such as radio waves or freely propagated electromagnetic waves, electromagnetic waves propagated via waveguide or other transmission media (such as optical pulses via fiber-optic cables), or electric signals propagated via electric wires.

The described computer-readable program instruction can be downloaded from the computer-readable storage medium to each computing/processing device, or to an external computer or external storage via Internet, local area network, wide area network and/or wireless network. The network can comprise copper-transmitted cable, optical fiber transmission, wireless transmission, router, firewall, switch, network gate computer and/or edge server. The network adapter card or network interface in each computing/processing device receives computer-readable program instructions from the network and forwards the computer-readable program instructions for storage in the computer-readable storage medium of each computing/processing device.

The computer program instructions for executing operations of the present disclosure can be assembly instructions, instructions of instruction set architecture (ISA), machine instructions, machine-related instructions, microcodes, firmware instructions, state setting data, or source codes or target codes written in any combinations of one or more programming languages, wherein the programming languages consist of object-oriented programming languages, e.g., Smalltalk, C++ and so on, and traditional procedural programming languages, such as “C” language or similar programming languages. The computer-readable program instructions can be implemented fully on the user computer, partially on the user computer, as an independent software package, partially on the user computer and partially on the remote computer, or completely on the remote computer or server. In the case where remote computer is involved, the remote computer can be connected to the user computer via any type of networks, comprising local area network (LAN) and wide area network (WAN), or to the external computer (e.g., connected via Internet using the Internet service provider). In some embodiments, state information of the computer-readable program instructions is used to customize an electronic circuit, e.g., programmable logic circuit, field programmable gate array (FPGA) or programmable logic array (PLA). The electronic circuit can execute computer-readable program instructions to implement various aspects of the present disclosure.

Various aspects of the present disclosure are described here with reference to flow chart and/or block diagram of method, apparatus (system) and computer program products according to embodiments of the present disclosure. It should be understood that each block of the flow chart and/or block diagram and the combination of various blocks in the flow chart and/or block diagram can be implemented by computer-readable program instructions.

The computer-readable program instructions can be provided to the processing unit of general-purpose computer, dedicated computer or other programmable data processing apparatuses to manufacture a machine, such that the instructions that, when executed by the processing unit of the computer or other programmable data processing apparatuses, generate an apparatus for implementing function/actions stipulated in one or more blocks in the flow chart and/or block diagram. The computer-readable program instructions can also be stored in the computer-readable storage medium and cause the computer, programmable data processing apparatus and/or other devices to work in a particular manner, such that the computer-readable medium stored with instructions contains an article of manufacture, comprising instructions for implementing various aspects of the function/actions stipulated in one or more blocks of the flow chart and/or block diagram.

The computer-readable program instructions can also be loaded into computer, other programmable data processing apparatuses or other devices, so as to execute a series of operation steps on the computer, other programmable data processing apparatuses or other devices to generate a computer-implemented procedure. Therefore, the instructions executed on the computer, other programmable data processing apparatuses or other devices implement function/actions stipulated in one or more blocks of the flow chart and/or block diagram.

The flow chart and block diagram in the drawings illustrate system architecture, function and operations that may be implemented by system, method and computer program product according to multiple implementations of the present disclosure. In this regard, each block in the flow chart or block diagram can represent a module, part of program segment or code, wherein the module and the part of program segment or code comprise one or more executable instructions for performing stipulated logic function. In some alternative implementations, it should be noted that the function indicated in the block can also take place in an order different from the one indicated in the drawings. For example, two successive blocks can be in fact executed in parallel or sometimes in a reverse order dependent on the involved function. It should also be noted that each block in the block diagram and/or flow chart and combinations of the blocks in the block diagram and/or flow chart can be implemented by a hardware-based system exclusive for executing stipulated function or actions, or by a combination of dedicated hardware and computer instructions.

Various implementations of the present disclosure have been described above and the above description is only exemplary rather than exhaustive and is not limited to the implementations of the present disclosure. Many modifications and alterations, without deviating from the scope and spirit of the explained various implementations, are obvious for those skilled in the art. The selection of terms in the text aims to best explain principles and actual applications of each implementation and technical improvements made in the market by each embodiment, or enable other ordinary skilled in the art to understand implementations of the present disclosure.

1. An information processing method, comprising:
receiving first information associated with an operation behavior of a medical testing device, the first information being associated with data collection performed by the medical testing device during operation; and
outputting at least part of the first information.

2. The method according to claim 1, wherein receiving the first information comprises receiving at least one of the following:
a set of locations of the medical testing device during the operation;
sequence information associated with the set of locations;
time information associated with the set of locations;
a trajectory of the medical testing device during the operation;
a recommended trajectory of the medical testing device during the operation;
a deviation degree of the trajectory from the recommended trajectory;
speed information of the medical testing device during the operation;
smoothness of the medical testing device during the operation;
image data collected by the medical testing device during the data collection;
image quality of the image data;
an organ image of an organ for which the operation is directed;
a degree of qualification of a to-be-tested part of the medical testing device to medical testing device examination;
a score of the operation behavior;
statistical information of the operation behavior;
comparative information of the statistical information;
suggestions of improvement to the operation behavior;
comparison of the different first information for the different operation behavior; and
the operation behavior determined according to the score and a score threshold.

3. The method of claim 2, wherein the statistical information comprises at least one of the following:
a quantity of image data collected by the operation behavior in at least part of the set of locations;
division of the operation behavior according to different operators of the medical testing device; and
division of the operation behavior according to organizations to which different operators of the medical testing device belong.

4. The method of claim 2, wherein outputting the at least part of the first information comprises at least one of the following:
outputting at least part of the set of locations in association with the organ image; and
outputting at least part of the trajectory in association with the organ image.

5. The method of claim 2, wherein outputting at least part of the first information comprises at least one of the following:
outputting the trajectory and the recommended trajectory in association; and
outputting the trajectory and the deviation degree in association.

6. The method of claim 1, wherein receiving the first information comprises:
receiving the first information from a cloud storage device, the first information being transmitted from a data analysis device of the medical testing device to the cloud storage device, and the first information being determined based on input data of the medical testing device.

7. The method of claim 1, wherein outputting the at least part of the first information comprises:
receiving an input instruction for the first information; and
determining the part of the first information in response to the input instruction.

8. The method of claim 7, wherein the input instruction further comprises a screening instruction, and the screening instruction comprises screening conditions comprising at least one of the following: an operator identification of an operator associated with an operation behavior of the medical testing device, an organization identification of an organization to which an operator of the medical testing device belongs, a date the operation behavior occurred, a time period during which the operation behavior occurred, a score threshold of scoring to the operation behavior, a degree of qualification threshold of a degree of qualification of a to-be-tested part of the medical testing device to medical testing device examination, a statistical information threshold of statistical information of the operation behavior and an image quality threshold.

9. The method of claim 1, wherein outputting the at least part of the first information comprises one of the following:
displaying the at least part of the first information; and
printing the at least part of the first information.

10. The method of claim 1, wherein outputting the at least part of the first information comprises: displaying the at least part of the first information in at least one of the following display manners:
multi-view switching display,
video display,
virtual reality display,
augmented reality display, and
3D display.

11. The method of claim 1, wherein outputting the at least part of the first information comprises:
selecting a display manner based on a type of the first information; and
displaying the at least part of the first information in the selected display manner.

12. The method of claim 1, further comprising:
receiving a first input indicating another display manner that is different from a current display manner of the at least part of the first information; and
displaying the at least part of the first information in the other display manner.

13. The method of claim 1, further comprising:
receiving a second input indicating to-be-displayed information; and
outputting the first information that matches the to-be-displayed information and not displayed in the first information.

14. An information processing method, comprising:
receiving, at the terminal device, first identification information associated with a medical device;
obtaining second identification information of an operator associated with the terminal device; and
associating the medical device with the operator based on the first identification information and the second identification information.

15. The method of claim 14, further comprising:
associating an operation performed by the operator through the medical device with the operator.

16. The method of claim 14, wherein receiving the first identification information comprises receiving the identification information by at least one of the following:
scanning a QR code corresponding to the identification information;
scanning a barcode corresponding to the identification information;
receiving an input of a device identification corresponding to the identification information;
receiving an audio input corresponding to the identification information;
receiving a video input corresponding to the identification information; and
receiving a tactile input corresponding to the identification information.

17. The method of claim 14, wherein the second identification information comprises at least one of the following:
a name of the operator,
a username of the operator,
a telephone number of the operator,
an image associated with the operator,
a job title of the operator.

18. The method of claim 14, further comprising:
if determining to cease use of the medical device, disassociating the medical device from the operator.

19. The method of claim 18, further comprising determining to cease use of the medical device according to at least one of:
receiving a request at a terminal device to stop using the medical device;
time duration that the terminal device associated with the medical device exceeding a threshold length;
the medical device entering a dormant state; and
the medical device entering a shutdown state.

20. An information processing method, comprising:
receiving, at the terminal device, first indication information from a user, the first indication information indicating at least one operation performed by a medical device;
receiving second indication information from the user, the second indication information indicating an operator of the at least one operation; and
associating the indicated at least one operation with the indicated operator.

21-22. (canceled)
